(19) United States
(12) Patent Application Publication
(10) Pub. No.: US 2018/0369325 A1
(43) Pub. Date: Dec. 27, 2018

(54) COMPOUNDS AND METHODS FOR TREATING METABOLIC DISORDERS

(71) Applicant: GARVAN INSTITUTE OF MEDICAL RESEARCH, Darlinghurst (AU)

(72) Inventors: Herbert HERZOG, Darlinghurst (AU); Paul BALDOCK, Darlinghurst (AU); Lei ZHANG, Darlinghurst (AU); Nikki LEE, Darlinghurst (AU)

(73) Assignee: GARVAN INSTITUTE OF MEDICAL RESEARCH, Darlinghurst (AU)

(21) Appl. No.: 15/777,510

(22) PCT Filed: Nov. 18, 2016
(86) PCT No.: PCT/AU2016/051112

§ 371 Date: May 18, 2018

(30) Foreign Application Priority Data
Nov. 19, 2015 (AU) 2015904770

(51) Int. Cl. A61K 38/17

(52) U.S. Cl. CPC A61K 38/17

(57) ABSTRACT

The present disclosure relates to methods and reagents for treating or preventing metabolic disorders, including, but not limited to, type 2 diabetes, obesity, hyperglycaemia and other conditions associated with an abnormality of glucose metabolism.

### TECHNICAL FIELD

The present disclosure relates to methods and reagents for treating or preventing metabolic disorders, including, but not limited to, type 2 diabetes, obesity, hyperglycaemia and other conditions associated with an abnormality of glucose metabolism.

### BACKGROUND

Maintenance of glucose homeostasis requires the coordination of glucose supply to a wide variety of tissues of differing energetic demands, whilst balancing endogenous gluconeogenesis with the periodic supply of glucose from the diet. Appropriate glucose supply is fundamental to effective and consistent cellular function, and can only be ensured through complex communication and feedback pathways regulating insulin-dependent and independent glucose uptake in target tissues.

Obesity and type 2 diabetes are common, chronic conditions associated with perturbations in glucose homeostasis, each having major health and economic impacts on society. Type 2 diabetes in particular is a serious health concern in more developed societies that ingest foodstuffs high in sugars and/or fats. The disease is associated with blindness, heart disease, stroke, kidney disease, hearing loss, gangrene and impotence. Type 2 diabetes and its complications are leading causes of premature death in the Western world. There are an estimated 23.6 million people in the United States (7.8% of the population) with diabetes with 17.9 million being diagnosed, 90% of whom suffer from type 2 diabetes. With prevalence rates doubling between 1990 and 2005, the Center for Disease Control (CDC) in USA has characterized the increase as an epidemic. Traditionally considered a disease of adults, type 2 diabetes is increasingly diagnosed in children in parallel to rising obesity rates due to alterations in dietary patterns as well as in life styles during childhood

Generally, type 2 diabetes adversely affects the way the body converts or utilizes ingested sugars and starches into glucose. The majority of overweight and obese individuals do not develop diabetes because their pancreatic β-cells adequately respond and prevent overt hyperglycaemia through increased insulin secretion. This is known as β-cell compensation. Those who progress to type 2 diabetes do so because insulin secretion cannot match insulin demand. In this regard, type 2 diabetes is typically associated with a progressive decline in β-cell function, which is manifest primarily as a selective loss of GSIS. There is now good evidence for reduced β-cell mass linked with increased rates of β-cell apoptosis in people suffering from type 2 diabetes relative to weight-matched subjects without diabetes.

In most type 2 diabetes subjects, the metabolic entry of glucose into various “peripheral” tissues is reduced and there is increased liberation of glucose into the circulation from the liver. Thus, there is an excess of extracellular glucose and a deficiency of intracellular glucose. Elevated blood lipids and lipoproteins are a further common complication of diabetes. The cumulative effect of these diabetes-associated abnormalities is severe damage to blood vessels and nerves.

Many available treatments for type 2 diabetes, some of which have not changed substantially in many years, have recognized limitations. For example, while physical exercise and reductions in dietary intake of fat, high glycemic carbohydrates, and calories can dramatically improve the diabetic condition, compliance with this treatment is very poor because of well-entrenched sedentary lifestyles and excess food consumption, especially of foods containing high amounts of saturated fat.

Conventional drug-based treatments for type 2 diabetes are very limited, and focus on attempting to control blood glucose levels to minimize or delay complications.

Current treatments target either insulin resistance (metformin, thiazolidinediones (“TZDs”)), or insulin release from the β-cells (sulphonylureas, exenatide). Sulphonylureas, and other compounds that act by depolarizing the β-cell, have the side effect of hypoglycemia since they cause insulin secretion independent of circulating glucose levels. Other side effects of current therapies include weight gain, loss in responsiveness to therapy over time, gastrointestinal problems, and edema.

One currently approved drug, Januvia (sitagliptin, a dipeptidyl peptidase IV (DPPIV) inhibitor) increases blood levels of incretin hormones (e.g., glucagon-like peptide (GLP)-1), which can increase insulin secretion, reduce glucagon secretion and have other less well characterized effects. However, Januvia and other DPPIV inhibitors may also influence the tissue levels of other hormones and peptides, and the long-term consequences of this broader effect have not been fully investigated. For example, DPPIV is a tumor suppressor, and inhibition of this enzyme may increase the risk of some cancers, e.g., non-small cell lung cancer.

The use of clinically available agents that increase intracellular availability of GLP-1, such as orally active DPPIV inhibitors or injectable GLP-1 analogs, are also limited as a result of relatively short half-life of these agents. This means that they require frequent administration.

Another approach considered for the treatment of type 2 diabetes is the use of general lipase inhibitors to prevent fat digestion to thereby control body weight and risk of this condition. However, a weakness of this approach is that general lipase inhibitors, such as, orlistat inhibit glucose stimulated insulin secretion (GSIS; Mulder et al., (2004) Diabetes 53:122-128). This is likely because they are required for appropriate glucose sensing in pancreatic β-cells.

It is clear from the foregoing that there is a need in the art for a method to treat or prevent or delay the onset or progression of abnormalities of glucose metabolism, e.g., type 2 diabetes.

Whilst not fully understood, emerging evidence suggests that there is a link between type 2 diabetes and osteoporosis, another major chronic condition affecting 2.2 million Australians. For example, insulin has been reported as acting on osteoblasts and, amongst other functions, has been shown to stimulate osteocalcin release from bone by stimulating bone resorption (Ferron et. al., (2010) Cell, 142:296-308; Fulzele et al., (2010) Cell, 142: 309-319). In patients with type 2 diabetes, bone formation, as well as bone microarchitectural integrity, are altered resulting in a higher risk of osteoporotic fractures and inadequate bone regeneration following injury.

One of the most novel and exciting findings in recent years is evidence that skeletal tissue also plays an active role in regulating whole body glucose homeostasis through the action of factors released from bone tissue. However, the regulation of glucose metabolism by bone has not been fully elucidated.

Identifying and targeting pathways originating in bone tissue which act directly to alter whole body glucose homeostasis by affecting insulin secretion and insulin action therefore has the potential for the development of new therapeutics for metabolic diseases such as obesity and type 2 diabetes.

### SUMMARY

The present disclosure is based on the inventors' finding that osteoglycin acts as a humoral factor which is capable of regulating insulin and glucose homeostasis. In particular, the inventors have shown that increasing the activity of osteoglycin improves glucose tolerance by increasing pancreatic insulin secretion in response to increasing blood glucose levels and by enhancing insulin action and sensitivity. For example, the inventors have demonstrated that mice administered osteoglycin prior to a glucose tolerance test (GTT) had improved glucose tolerance, yet exhibited a dose-dependent reduction in insulin levels. On the other hand, the inventors have demonstrated that osteoglycin knockout (Ogn^{-/-}) mice have significantly impaired glucose tolerance under chow and high fat diet (HFD) conditions resulting in higher glucose and associated insulin levels. The inventors also observed significantly lower circulating levels of osteoglycin in human subjects with type 2 diabetes as compared to lean and obese/overweight but insulin sensitive control subjects. These findings provide the basis of inducing or improving GSIS and/or treating or preventing a glucose metabolism disorder using agents which increase osteoglycin activity. These findings also provide the basis for supplementing existing therapies for glucose metabolism disorders.

The present disclosure provides a method of treating or preventing an abnormality of glucose metabolism in a subject, said method comprising increasing the activity of osteoglycin in the subject.

The present disclosure provides a method for increasing glucose stimulated insulin secretion (GSIS) in a subject having reduced or impaired GSIS, said method comprising increasing the activity of osteoglycin in the subject. A subject having reduced or impaired GSIS can be readily determined by a medical practitioner based on accepted criteria at the time e.g., as advised by the World Health Organisation or national body (such as American Diabetes Association, National Health and Medical Research Council Australia).

In one example, the increase in activity of osteoglycin is sufficient to enhance insulin action in the subject and thereby improve glucose tolerance of the subject.

In one example, the increase in activity of osteoglycin is sufficient to increase insulin secretion from pancreatic beta cells in the subject in response to an increase in glucose levels in the subject.

In one example, the subject on whom the method is to be performed may suffer from an abnormality of glucose metabolism. Exemplary conditions characterised by an abnormality of glucose metabolism are selected from the group consisting of type 2 diabetes, obesity, hyperglycaemia and combinations thereof.

In one example, the subject may suffer from type 2 diabetes or is at risk of developing type 2 diabetes.

In one example, the increase in osteoglycin activity is achieved by administering one or more agents select from:
(i) osteoglycin or a functional fragment, analog or derivative thereof to the subject; and/or
(ii) an expression vector comprising a nucleic acid encoding osteoglycin or a functional fragment thereof, wherein said expression vector is capable of expressing osteoglycin or a functional fragment thereof in the subject.

In one example, the agent for increasing osteoglycin activity may be osteoglycin protein e.g., a full length osteoglycin protein. In another example, the agent for increasing osteoglycin activity may be a functional fragment of a full length osteoglycin protein. In another example, the agent for increasing osteoglycin activity may be an analog of an osteoglycin protein or fragment thereof. In another example, the agent for increasing osteoglycin activity may be an derivative of an osteoglycin protein or fragment thereof. In another example, the agent for increasing osteoglycin activity may be an expression vector comprising a nucleic acid encoding osteoglycin or a functional fragment thereof e.g., a plasmid or viral vector.

In one example, the agent(s) is/are administered in the form of a pharmaceutical composition. Suitable administration regimes will be known in the art and will vary according to the condition to be treated. However, in an exemplary regime, the pharmaceutical composition is administered a plurality of times to thereby maintain glucose homeostasis in the subject.

A pharmaceutical composition for use in the method of the disclosure may comprise, or may be administered concurrently or concomitantly with, another therapeutic compound for treatment of a metabolic condition. Exemplary therapeutic compounds for treatment of a metabolic conditions may be selected from the group consisting of a glucagon like peptide 1 (GLP-1), a GLP-1 analog, a GLP-1 receptor agonist, a dipeptidyl peptidase 4 (DPPIV) inhibitor, a sulphonurea, a meglitinide, a GPR40 agonist, a GPR119 agonist, a sodium glucose co-transporter-2 inhibitor, a thiazolidinone, metformin, a glucokinase activator and an insulin analog.

For example, the GLP-1 analog or GLP-1 receptor agonist may be selected from the group consisting of exenatide, liraglutide, exenatide LAR, taspoglutide, albiglutide, dulaglutide and GLP1 conjugated to albumin. For example, the DPPIV inhibitor may be selected from the group consisting of sitagliptin (JANUVIA) and vidagliptin (GALVUS). For example, the sulphonylurea may be selected from the group consisting of glibenclamide, glyburide and gliclazide. For example, the meglitinide may be selected from the group consisting of repaglinide and nateglinide. For example, the GPR40 agonist may be selected from the group consisting of TAK-875 and AMG-837. For example, the GPR119 agonist may be selected from the group consisting of PSN632408, JNJ-38431055. For example, the glucokinase activator may be selected from the group consisting of GKA50, piragliatin (RO4389620) and ZYGK1. For example, the sodium glucose co-transporter-2 inhibitor is empagliflozin. For example, the thiazolidinone is rosiglitazone, pioglitazone or troglitazone. For example, the insulin analog is insulin lispro, insulin aspart, insulin glulisine, insulin detemir, insulin degludec, insulin glargine or NPH insulin.

The present disclosure also provides for use of agent which increases activity of osteoglycin in a subject in the preparation of a medicament for treatment or prevention of abnormal glucose metabolism in a subject in need thereof.

The present disclosure also provides for use of agent which increases activity of osteoglycin in the preparation of a medicament for treatment or prevention of a condition associated with reduced or impaired glucose-stimulated insulin secretion (GSIS).

In one example, the agent is present in the medicament in an amount effective to enhance insulin action in the subject and thereby improve glucose tolerance of the subject. For example, the agent may be present in the medicament in an amount effective to increase insulin secretion from pancreatic beta cells in the subject in response to an increase in glucose levels.

Exemplary conditions for which the medicament is suitable for treatment or prevention may be selected from the group consisting of type 2 diabetes, obesity, hyperglycaemia and combinations thereof.

In one example, the medicament is for treatment or prevention of type 2 diabetes.

In one example, the agent in the medicament which increases osteoglycin activity is select from:
(i) osteoglycin or a functional fragment, analog or derivative thereof; and/or
(ii) an expression vector comprising a nucleic acid encoding osteoglycin or a functional fragment thereof, wherein said expression vector is capable of expressing osteoglycin or a functional fragment thereof in a cell.

For example, the agent for increasing osteoglycin activity may be osteoglycinprotein e.g., a full length osteoglycin protein. In another example, the agent for increasing osteoglycin activity may be a functional fragment of a full length osteoglycin protein. In another example, the agent for increasing osteoglycin activity may be an analog of an osteoglycin protein or fragment thereof. In another example, the agent for increasing osteoglycin activity may be an derivative of an osteoglycin protein or fragment thereof. In another example, the agent for increasing osteoglycin activity may be an expression vector comprising a nucleic acid encoding osteoglycin or a functional fragment thereof e.g., a plasmid or viral vector.

In one example, the medicament comprises another therapeutic compound for treatment of a metabolic condition. For example, the medicament may comprise one or more of a glucagon like peptide 1 (GLP-1), a GLP-1 analog, a GLP-1 receptor agonist, a dipeptidyl peptidase 4 (DPPIV) inhibitor, a sulphonurea, a meglitinide, a GPR40 agonist, a GPR119 agonist, a sodium glucose co-transporter-2 inhibitor, a thiazolidinone, metformin, a glucokinase activator or an insulin analog.

For example, the GLP-1 analog or GLP-1 receptor agonist may be selected from the group consisting of exenatide, liraglutide, exenatide LAR, taspoglutide, albiglutide, dulaglutide and GLP1 conjugated to albumin. For example, the DPPIV inhibitor may be selected from the group consisting of sitagliptin (JANUVIA) and vidagliptin (GALVUS). For example, the sulphonylurea may be selected from the group consisting of glibenclamide, glyburide and gliclazide. For example, the meglitinide may be selected from the group consisting of repaglinide and nateglinide. For example, the GPR40 agonist may be selected from the group consisting of TAK-875 and AMG-837. For example, the GPR119 agonist may be selected from the group consisting of PSN632408, JNJ-38431055. For example, the glucokinase activator may be selected from the group consisting of GKA50, piragliatin (RO4389620) and ZYGK1. For example, the sodium glucose co-transporter-2 inhibitor is empagliflozin. For example, the thiazolidinone is rosiglitazone, pioglitazone or troglitazone. For example, the insulin analog is insulin lispro, insulin aspart, insulin glulisine, insulin detemir, insulin degludec, insulin glargine or NPH insulin.

The present disclosure also provides a composition comprising an agent which increases activity of osteoglycin for treatment or prevention of abnormal glucose metabolism in a subject in need thereof.

The present disclosure also provides composition comprising an agent which increases activity of osteoglycin for treatment or prevention of a condition associated with reduced or impaired glucose-stimulated insulin secretion (GSIS).

In one example, the agent which increases activity of osteoglycin is present in the composition in an amount effective to enhance insulin action in the subject and thereby improve glucose tolerance of the subject.

In one example, the agent which increases activity of osteoglycin is present in the composition in an amount effective to increase insulin secretion from pancreatic beta cells in the subject in response to an increase in glucose levels.

In one example, the composition described herein is for treatment or prevention of a condition selected from the group consisting of type 2 diabetes, obesity, hyperglycaemia and combinations thereof. An exemplary composition is for treatment or prevention of type 2 diabetes.

In one example, the composition comprises an agent which increases osteoglycin activity select from:
(i) osteoglycin or a functional fragment, analog or derivative thereof; and/or
(ii) an expression vector comprising a nucleic acid encoding osteoglycin or a functional fragment thereof, wherein said expression vector is capable of expressing osteoglycin or a functional fragment thereof in a cell.

For example, the agent for increasing osteoglycin activity may be osteoglycin protein e.g., a full length osteoglycin protein. In another example, the agent for increasing osteoglycin activity may be a functional fragment of a full length osteoglycin protein. In another example, the agent for increasing osteoglycin activity may be an analog of an osteoglycin protein or fragment thereof. In another example, the agent for increasing osteoglycin activity may be an derivative of an osteoglycin protein or fragment thereof. In another example, the agent for increasing osteoglycin activity may be an expression vector comprising a nucleic acid encoding osteoglycin or a functional fragment thereof e.g., a plasmid or viral vector.

In one example, a composition disclosed herein may comprise a pharmaceutically acceptable carrier, diluent or excipient.

In another example, a composition disclosed herein may comprise another therapeutic compound for treatment of a metabolic condition. For example, the composition may comprise one or more of a glucagon like peptide 1 (GLP-1), a GLP-1 analog, a GLP-1 receptor agonist, a dipeptidyl peptidase 4 (DPPIV) inhibitor, a sulphonurea, a meglitinide, a GPR40 agonist, a GPR119 agonist, a sodium glucose co-transporter-2 inhibitor, a thiazolidinone, metformin, a glucokinase activator or an insulin analog.

For example, the GLP-1 analog or GLP-1 receptor agonist may be selected from the group consisting of exenatide, liraglutide, exenatide LAR, taspoglutide, albiglutide, dulaglutide and GLP1 conjugated to albumin. For example, the DPPIV inhibitor may be selected from the group consisting of sitagliptin (JANUVIA) and vidagliptin (GALVUS). For example, the sulphonylurea may be selected from the group consisting of glibenclamide, glyburide and gliclazide. For example, the meglitinide may be selected from the group consisting of repaglinide and nateglinide. For example, the GPR40 agonist may be selected from the group consisting of TAK-875 and AMG-837. For example, the GPR119 agonist may be selected from the group consisting of PSN632408, JNJ-38431055. For example, the glucokinase activator may be selected from the group consisting of GKA50, piragliatin (RO4389620) and ZYGK1. For example, the sodium glucose co-transporter-2 inhibitor is empagliflozin. For example, the thiazolidinone is rosiglitazone, pioglitazone or troglitazone. For example, the insulin analog is insulin lispro, insulin aspart, insulin glulisine, insulin detemir, insulin degludec, insulin glargine or NPH insulin.

### BRIEF DESCRIPTION OF DRAWINGS

The following figures form part of the present specification and are included to further demonstrate certain aspects of the present disclosure. The disclosure may be better understood by reference to one or more of these figures in combination with the detailed description of specific embodiments presented herein.

FIG. 1 provides scatter plots from osteoblast cells isolated from (A) non-GFP expressing mice and (B) GFP-expressing mice, each showing GFP signal, forward scatter (FSC) and cell count. Green GFP positive (GFP POS) gate represents the purified osteoblast cells that were selected for further analysis.

FIG. 2 illustrates dose dependent (A) reductions in blood glucose levels, (B) area under the glucose curves, (c) serum insulin levels, and (D) area under the insulin curves, obtained from glucose tolerance tests performed on C57BL6 mice administered different doses of osteoglycin (Ogn) by introperitoneal injection.

FIG. 3 illustrates the level of insulin secretion by primary mouse pancreatic islet cells treated with and without osteoglycin in vitro in response to low and high glucose levels. Data are means±SEM of 5-7 per group from a single experiment.

FIG. 4 shows levels of Ins1 and Ins2 mRNA expression in MIN6 cells treated with different doses of osteoglycin, as well as in the absence of osteoglycin. Data are means±SEM of 8 per group from two experiments. *=p<0.05

FIG. 5 show the level of Akt phosphorylation (Ser473) in quadricept muscles of mice treated with osteoglycin (3 μg) or PBS by intraperitoneal injection followed by either saline or insulin (0.5 IU/kg) 4 hours later: (A) shows the pAkt (Ser473) to Akt ratios of the respective treatment groups relative to that of untreated groups; and (B) shows representative immunoblots for each of the respective groups. Data are means±SEM of 4-6 mice per group. *=p<0.05 as indicated.

FIG. 6 shows (A) lower serum levels of osteoglycin and (B) higher fasting serum glucose levels, in widltype mice, heterozygous Ogn knockout mice, and homozygous Ogn knockout mice, respectively. Data are means±SEM of 4-6 mice per group. *=p<0.05 and **=p<0.01

FIG. 7 illustrates that (A) male homozygous Ogn^{-/-} mice have significantly lower body weight and lean mass, (B) male homozygous Ogn^{-/-} mice have a trend towards impaired clearance of glucose during a glucose tolerance test (GTT) compared to heterozygous Ogn^{+/-} mice, (C) female homozygous Ogn^{-/-} mice display significantly higher glucose levels as a percentage of basal levels during a GTT relative to heterozygous controls, and (D) male homozygous Ogn^{-/-} mice display significantly higher glucose levels as a percentage of basal levels during a GTT relative to heterozygous controls. Data are means±SEM of 3-6 mice per group. *=p<0.05.

FIG. 8 illustrates that Ogn^{-/-} mice had significantly impaired glucose clearance as compared to wildtype littermates during a glucose tolerance test (GTT) under both chow and high fat diet (HFD) fed conditions, as shown by (A) higher glucose levels throughout the GTT and (B) higher glucose levels expressed as area under the curve. n=8-12 mice per group. *=p<0.05, **=p<0.01, ***=p<0.001.

FIG. 9 illustrates that Ogn^{-/-} mice had higher insulin levels under both chow and high fat diet (HFD) fed conditions (A) throughout the glucose tolerance test (GTT) and (B) expressed as area under the curve. n=8-12 mice per group. *=p<0.05, **=p<0.01, ***=p<0.001.

FIG. 10 shows that there was no difference in body weight between Ogn^{-/-} mice and wildtype littermates at 14-16 weeks of age. n=8-12 mice per group.

FIG. 11 shows that there was no difference in fat mass between Ogn^{-/-} mice and wildtype littermates at 14-16 weeks of age. n=8-12 mice per group.

FIG. 12 shows that Ogn^{-/-} mice had significantly increased lean mass as compared to wildtype littermates at 14-16 weeks of age. n=8-12 mice per group. *=p<0.05.

FIG. 13 shows that Ogn mice had significantly increased femur (A) bone mineral density (BMD) and (B) bone mineral content (BMC) compared to wildtype littermates at 14-16 weeks of age, as measured by dual x-ray densitometry (DXA). n=8-12 mice per group. *=p<0.05.

FIG. 14 illustrates that Ogn^{-/-} mice showed significant increases in both cortical and trabecular bone in the femur as determined by micro cT analysis. Specifically, Ogn^{-/-} mice showed significant increases in (A) cortical bone volume (BV) and (B) periosteal perimeter, with no change in (C) cortical thickness and significant increases in (D) trabecular bone volume (BV/TV) and (E) trabecular thickness, with no change in (F) trabecular number, as compared to wildtype littermates at 14-16 weeks of age. n=8-12 mice per group. *=p<0.05, **=p<0.01, and ***=p<0.001.

FIG. 15 illustrates that circulating levels of osteoglycin are significantly lower in patients with type 2 diabetes (T2D) as compared to both lean and obese/overweight but insulin sensitive controls.

FIG. 16 shows a decrease in circulating levels of osteoglycin in 16 week old male wildtype (WT) mice which have been fed a high fat diet (HFD) for 8 weeks compared to those fed chow.

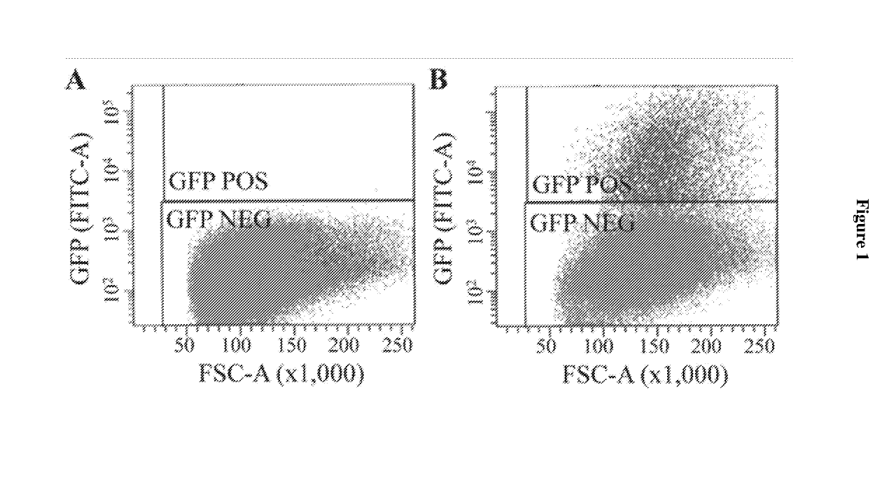

### KEY TO THE SEQUENCE LISTING

SEQ ID NO: 1: Amino acid sequence for human osteoglycin protein.
SEQ ID NO: 2: DNA sequence encoding human osteoglycin protein.
SEQ ID NO: 3: Amino acid sequence for GLP-1.
SEQ ID NO: 4: Amino acid sequence for Exenatide.
SEQ ID NO: 5: Amino acid sequence for Liraglutide
SEQ ID NO: 6: Amino acid sequence for Taspoglutide.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each example of the disclosure described herein is to be applied mutatis mutandis to each and every other example unless specifically stated otherwise.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure, as described herein.

The present disclosure is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in Sambrook, Fritsch & Maniatis, Molecular Cloning: A Laboratory Manual, Cold Spring Harbor Laboratories, New York, Second Edition (1989), whole of Vols I, II, and III; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, pp1-22; Atkinson et al, pp35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series; J. F. Ramalho Ortigao, “The Chemistry of Peptide Synthesis” In: Knowledge database of Access to Virtual Laboratory website (Interactiva, Germany); Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, J. eds.), vol. 2, pp. 1-284, Academic Press, New York. 12. Wunsch, E., ed. (1974) Synthese von Peptiden in Houben-Weyls Metoden der Organischen Chemie (Müller, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); and Animal Cell Culture: Practical Approach, Third Edition (John R. W. Masters, ed., 2000), ISBN 0199637970, whole of text.

As used herein, the singular forms of “a”, “and” and “the” include plural forms of these words, unless the context clearly dictates otherwise.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Selected Definitions

For the purposes of nomenclature only and not limitation, a sequence of a human osteoglycin protein is set forth in SEQ ID NO: 1. Similarly, for the purposes of nomenclature only and not limitation, a nucleic acid sequence encoding a human osteoglycin protein is set forth in SEQ ID NO: 2. In one example, a reference herein to “osteoglycin” is a reference to human osteoglycin. Sequences of human osteoglycin are also set forth in NCBI RefSeqs NP_054776 and NM_014057.

As used herein, the term “abnormality of glucose metabolism” shall be taken to mean a condition characterised by hyperglycemia and/or β-islet cell dysfunction. For example, the abnormality of glucose metabolism may be type 2 diabetes.

The term “increasing the activity of osteoglycin”, or similar, as used herein may refer to the ability of an agent to enhance or increase one or more biological activities of osteoglycin in a subject. The osteoglycin may be endogenous or exogenous to the subject. Alternatively, or in addition, the term “increasing the activity of osteoglycin”, or similar, may refer to an ability of an agent to increase an amount or level of osteoglycin in the subject. For example, the agent may be or may comprise ostoeglycin or an analog thereof, or may act by increasing expression of osteoglycin in the subject.

As described herein, an exemplary activity of osteoglycin is its ability to enhance insulin action and/or sensitivity and/or increase insulin secretion from pancreatic beta cells in response to an increase in blood glucose levels. Accordingly, in one example, an increase in the activity of osteoglycin may be detected as an enhancement of insulin action in a cell or subject. In another example, an increase in the activity of osteoglycin may be detected as an increase in insulin secretion from pancreatic beta cells in response to an increase in blood glucose levels.

As used herein, the term “improving glucose tolerance” refers to improving the ability of a subject to control the level of glucose in the bloodstream via modifying secretion, sensitivity and/or activity of insulin in the subject, and thereby maintain blood-glucose in a normal range. For example, in a subject who produces lower than normal amounts of insulin, “improving glucose tolerance” may involve improving insulin action and/or sensitivity such that less insulin is required to maintain blood-glucose in a normal range. For example, in a subject who produces insufficient amounts of insulin, “improving glucose tolerance” may involve enhancing or increasing insulin secretion by pancreatic beta cells in response to increasing blood glucose levels, such that an appropriate amount of insulin is available in the subject to transport circulating glucose from the bloodstream to cells For example, in a subject who is insulin resistant, “improving glucose tolerance” may involve improving insulin sensitivity such that cells are more sensitive to circulating insulin and thereby able to transport glucose from blood to cells.

Reference herein to an “analog” of osteoglycin will be understood to mean a osteoglycin polypeptide or protein that is modified to comprise one or more naturally-occurring and/or non-naturally-occurring amino acids, provided that the analog displays the activity of osteoglycin e.g., enhancing insulin action and/or increasing insulin secretion from pancreatic beta cells in a subject in response to an increase in glucose levels e.g., as determined by a method known in the art and/or described herein. For example, an “analog” of osteoglycin encompasses an osteoglycin protein or polypeptide comprising one or more conservative amino acid changes. The term “analog” also encompasses an osteoglycin protein or polypeptide comprising, for example, one or more D-amino acids.

Reference herein to a “derivative” will be understood to include a polypeptide or protein that is derived from an osteoglycin protein or polypeptide. The term “derivative” encompasses fusion proteins comprising an osteoglycin protein or polypeptide or analog thereof. For example, the fusion protein may comprise a label, such as, for example, an epitope, e.g., a FLAG epitope or a V5 epitope. Such a tag is useful for, for example, purifying the fusion protein. Additional suitable fusion proteins will be apparent to the skilled artisan based on the disclosure herein. The term “derivative” also encompasses a derivatized osteoglycin protein or polypeptide, such as, for example, an osteoglycin protein or polypeptide modified to contain one or more-chemical moieties other than an amino acid. The chemical moiety may be linked covalently to the protein or polypeptide e.g., via an amino terminal amino acid residue, a carboxy terminal amino acid residue, or at an internal amino acid residue. Such modifications include the addition of a protective or capping group on a reactive moiety in the polypeptide, addition of a detectable marker or compound, and other changes that do not adversely destroy the activity of the protein or polypeptide.

As used herein the term “functional fragment” shall be taken to mean a fragment of an osteogylcin protein or derivative or analog thereof that has one or more biological activities of osteoglycin in a subject. As described herein, an exemplary activity of osteoglycin is an ability to enhance insulin action and/or sensitivity and/or increase insulin secretion from pancreatic beta cells in response to an increase in blood glucose levels. It will be appreciated that the activity of a functional fragment need not be equivalent to the full length osteoglycin protein, or analog or derivative thereof, from which it is derived. For example, the fragment may have slightly enhanced or reduced activity compared to the full length osteoglycin protein, or analog or derivative thereof, from which it is derived by virtue of the removal of flanking sequence.

As used herein, the term “glucose homeostasis” refers to the state in which blood glucose levels are maintained within a normal range by the body.

Reference herein to “concurrent” administration of two agents will be understood to mean that the agents are administered together or the same time. This does not mean that the agents are administered in the same solution or simultaneously.

Reference herein to “concomitant” administration of two agents will be understood to mean that the agents are administered one after the other, such that both agents are simultaneously active in a subject for a period of time.

As used herein, the terms “preventing”, “prevent” or “prevention” in the context of preventing a condition include administering an amount of an agent described herein sufficient to stop or hinder the development of at least one symptom of a specified disease or condition.

As used herein, the terms “treating”, “treat” or “treatment” include administering a therapeutically effective amount of an agent described herein sufficient to reduce or eliminate at least one symptom of a specified disease or condition.

As used herein, the term “subject” shall be taken to mean any animal, for example a mammalian animal. Exemplary mammals include a human or primate. In a particularly preferred example, the mammalian subject is a human.

### Agents for Increasing Osteoglycin Activity

In accordance with the present disclosure, an agent for increasing osteoglycin activity in a cell or subject may be selected from any one or more of an osteoglycin protein, and/or a functional fragment, analog or derivative thereof and/or an expression vector capable of expressing osteoglycin or a functional fragment thereof in a mammalian cell.

### Osteoglycin Protein, Fragments and Variants

In one example, the agent for increasing osteoglycin activity in a cell is an osteoglycin protein or functional fragment thereof. Osteoglycin (OGN), also referred to as OG, osteoinducive factor (OIF), mimecan, mimican and SLRR3A, is a small proteoglycan containing tandem leucine-rich repeats (LRRs) which is encoded by the OGN gene. An exemplary protein sequence for human osteoglycin is set forth in NCBI RefSeqs NP_054776 and in SEQ ID NO: 1 herein.

Accordingly, in one example, an osteoglycin protein comprising a sequence set forth in SEQ ID NO: 1 is contemplated for use in the methods of the present disclosure. Alternative splice variants of the osteoglycin protein set forth in SEQ ID NO: 1 which have osteoglycin activity are also contemplated for use in the method of the disclosure. In another example, the osteoglycin protein of the disclosure comprises a sequence at least about 75% or 80% or 85% or 90% or 95% or 97% or 98% or 99% identical to the sequence set forth in SEQ ID NO: 1, provided that the protein retains an activity of the osteoglycin protein set forth in SEQ ID NO: 1, as described herein. Furthermore, as osteoglycin is well conserved among mammalian species, the present disclosure also contemplates the use of an osteoglycin protein from a mammalian species. For example, the osteoglycin protein may be a murine, a bovine, a canine, an equine, a feline, a porcine, a simian or other mammalian osteoglycin protein.

Functional fragments, analogs and/or derivatives of the sequence set forth in SEQ ID NO: 1 are also contemplated for use in the methods of the present disclosure.

For example, the present disclosure contemplates the use of variants of an osteoglycin protein sequence set forth in SEQ ID NO: 1 comprising conservative substitutions or mutations in a small number of residues of the protein, provided that the variant retains the activity or function of the base osteoglycin protein. In one example, the osteoglycin variant of the disclosure comprises a sequence at least about 75% or 80% or 85% or 90% or 95% or 97% or 98% or 99% identical to the sequence set forth in SEQ ID NO: 1, wherein the protein retains an activity of osteoglycin protein set forth in SEQ ID NO: 1 i.e., an ability to enhance insulin action and/or sensitivity and/or increasing insulin secretion from pancreatic beta cells in response to an increase in glucose levels e.g., as determined by a method known in the art and/or described herein. A “conservative amino acid substitution” is one in which the amino acid residue is replaced with an amino acid residue having a similar side chain and/or hydropathicity and/or hydrophilicity.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Hydropathic indices are described, for example in Kyte and Doolittle J. Mol. Biol., 157: 105-132, 1982 and hydrophylic indices are described in, e.g., U.S. Pat. No. 4,554,101.

The present disclosure also contemplates the use of variants of an osteoglycin protein sequence set forth in SEQ ID NO: 1 comprising non-conservative substitutions or mutations in a small number of residues of the protein, provided that the variant retains the activity or function of the wild-type osteoglycin protein. For example, of particular interest are substitutions of charged amino acids with another charged amino acid and with neutral or positively charged amino acids. In some examples, the osteoglycin variant of the disclosure comprises 10 or fewer, e.g., 9 or 8 or 7 or 6 or 5 or 4 or 3 or 2 or 1 non-conservative amino acid substitutions.

The present disclosure also contemplates the use of variants of an osteoglycin protein sequence set forth in SEQ ID NO: 1 comprising one or more non-naturally occurring amino acids or amino acid analogues. For example, an osteoglycin protein variant may comprise one or more naturally occurring non-genetically encoded L-amino acids, synthetic L-amino acids or D-enantiomers of an amino acid. For example, the osteoglycin protein may comprise only D-amino acids. Exemplary non-coded amino acids include: hydroxyproline, β-alanine, 2,3-diaminopropionic acid, α-aminoisobutyric acid, N-methylglycine (sarcosine), ornithine, citrulline, t-butylalanine, t-butylglycine, N-methylisoleucine, phenylglycine, cyclohexylalanine, norleucine, naphthylalanine, pyridylananine 3-benzothienyl alanine 4-chlorophenylalanine, 2-fluorophenylalanine, 3-fluorophenylalanine, 4-fluorophenylalanine, penicillamine, 1,2,3,4-tetrahydro-tic isoquinoline-3-carboxylic acid β-2-thienylalanine, methionine sulfoxide, homoarginine, N-acetyl lysine, 2,4-diamino butyric acid, ρ-aminophenylalanine, N-methylvaline, homocysteine, homoserine, ε-amino hexanoic acid, δ-amino valeric acid, 2,3-diaminobutyric acid.

The osteoglycin protein or fragment, analog or derivative thereof may be provided as a conjugate. For example, the osteoglycin protein, fragment, analog or derivative may be conjugated to a nonproteinaceous moiety known in the art for improving one or more pharmacokinetic properties of protein-based agents. Preferably, the moiety suitable for derivatization of the protein, fragment, analog or derivative is a physiologically acceptable polymer, preferably a water soluble polymer. Such polymers are useful for increasing stability and/or reducing clearance (e.g., by the kidney) and/or for reducing immunogenicity of a protein, fragment, analog or derivative of the disclosure. Non-limiting examples of water soluble polymers include, but are not limited to, polyethylene glycol (PEG), polyvinyl alcohol (PVA), or propropylene glycol (PPG).

Alternatively, or in addition, the osteoglycin protein, fragment, analog or derivative described herein may be conjugated or linked to another protein, including another protein of the disclosure or an analog or derivative derived therefrom e.g., described herein. Other proteins are not excluded. Additional proteins will be apparent to the skilled artisan and include, for example, an immunomodulator or a half-life extending protein or a peptide or other protein that binds to serum albumin amongst others. When linked to another protein, the osteoglycin protein, fragment, analog or derivative described herein may a fusion protein or chimeric protein. Methods for producing fusion proteins and chimeric proteins are known in the art.

Conjugates of the osteoglycin protein, fragment, analog or derivative and additional moieties can be made using a variety of bifunctional protein-coupling agents such as, but not limited to, 4-(4′acetylphenoxy)butanoic acid (AcBut), 3-acetylphenyl acidic acid (AcPac), 4-mercapto-4-methyl-pentanoic acid (Amide), N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene), and derivatives thereof.

The osteoglycin protein, fragment, analog or derivative can be produced by recombinant means or synthetic means. Alternatively, the osteoglycin protein or protein fragments may be purified from a native source e.g., tissue.

In one example, the osteoglycin protein, fragment, analog or derivative is recombinant, and thus, will be produced by recombinant means.

Recombinant means generally comprise operably linking a nucleic acid encoding the protein to be expressed to a promoter to thereby form an expression construct, which can be an expression vector (e.g., a plasmid or phagemid). The present disclosure contemplates such an expression construct. The nucleic acid can be produced and/or isolated and cloned into an appropriate construct using methods known in the art and/or described in Ausubel et al (In: Current Protocols in Molecular Biology. Wiley Interscience, ISBN 047 150338, 1987) and/or (Sambrook et al (In: Molecular Cloning: Molecular Cloning: A Laboratory Manual, Cold Spring Harbor Laboratories, New York, Third Edition 2001).

Suitable promoters and/or expression vectors will be apparent to the skilled artisan based on the cell/expression system to be used. For example, typical promoters suitable for expression in a mammalian cell include, for example a promoter selected from the group consisting of, retroviral LTR elements, the SV40 early promoter, the SV40 late promoter, the CMV IE (cytomegalovirus immediate early) promoter, the EF_{1α} promoter (from human elongation factor 1α), the EM7 promoter, the UbC promoter (from human ubiquitin C). Examples of useful mammalian host cell lines include monkey kidney CV1 line transformed by SV40 (COS-7); human embryonic kidney line (293 or 293 cells) ; baby hamster kidney cells (BHK,); Chinese hamster ovary cells (CHO); African green monkey kidney cells (VERO-76); or myeloma cells (e.g., NS/0 cells). For example, the cells are CHO cells.

Other elements of expression constructs/vectors are known in the art and include, for example, enhancers, transcriptional terminators, polyadenylation sequences, nucleic acids encoding selectable or detectable markers and origins of replication.

Of course, the present disclosure contemplates expression of ostoeglycin protein, fragment, analog or derivative thereof in any cell, including bacterial cells, fungal cells, insect cells or plant cells.

Following production of a suitable expression construct, it is introduced into a suitable cell using any method known in the art. Exemplary methods include microinjection, transfection mediated by DEAE-dextran, transfection mediated by liposomes such as by using lipofectamine (Gibco, MD, USA) and/or cellfectin (Gibco, Md., USA), PEG-mediated DNA uptake, electroporation and microparticle bombardment such as by using DNA-coated tungsten or gold particles (Agracetus Inc., Wis., USA) amongst others.

The present disclosure also encompasses recombinant cells expressing an ostoeglycin protein, fragment, analog or derivative. The cells are then cultured under conditions known in the art to produce the ostoeglycin protein, fragment, analog or derivative.

In one example, the ostoeglycin protein, fragment, analog or derivative is synthetic, and may therefore be produced using chemical methods known to the skilled artisan.

For example, synthetic protein, fragment, analog or derivative may be prepared using known techniques of solid phase, liquid phase, or peptide condensation, or any combination thereof, and can include natural and/or unnatural amino acids. Amino acids used for polypeptide synthesis may be standard Boc (Na-amino protected Na-t-butyloxycarbonyl) amino acid resin with the deprotecting, neutralization, coupling and wash protocols of the original solid phase procedure of Merrifield, J. Am. Chem. Soc., 85:2149-2154, 1963, or the base-labile Nα-amino protected 9-fluorenylmethoxycarbonyl (Fmoc) amino acids described by Carpino and Han, J. Org. Chem., 37:3403-3409, 1972.

As the full length human osteoglycin protein is 298 amino acids in length, convergent or stepwise synthesis techniques are also contemplated for synthesizing the ostoeglycin protein, fragment, analog or derivative of the disclosure e.g., as described in Muir et al. (1993) Curr. Opin. Biotech., 4:420 and Miller et al., (1989) Science, 245:1149.

Following production/expression/synthesis, the ostoeglycin protein, fragment, analog or derivative may be purified using a method known in the art. Such purification provides the protein, fragment, analog or derivative substantially free of conspecific protein, nucleic acids, lipids, carbohydrates, and the like. For example, the protein, fragment, analog or derivative will be in a preparation wherein more than about 90% (e.g. 95%, 98% or 99%) of the protein in the preparation is ostoeglycin protein, fragment, analog or derivative.

Standard methods of purification are employed to obtain an isolated osteoglycin protein or functional fragment, analog or derivative thereof, including but not limited to various high-pressure (or performance) liquid chromatography (HPLC) and non-HPLC peptide isolation protocols, such as size exclusion chromatography, ion exchange chromatography, phase separation methods, electrophoretic separations, precipitation methods, salting in/out methods, immunochromatography, and/or other methods.

### Expression Constructs/Vectors

In one example, the agent for increasing osteoglycin activity is an expression construct or vector comprising a nucleic acid encoding an osteoglycin protein or fragment thereof, which is capable of expressing osteoglycin or the functional fragment thereof in a cell e.g., a mammalian cell.

For example, the expression construct or vector may comprise a nucleic acid encoding the osteoglycin protein sequence set forth in SEQ ID NO: 1. Alternatively, the expression construct or vector may comprise a nucleic acid encoding a variant of the sequence set forth in SEQ ID NO: 1 as described herein. For example, the expression construct or vector may comprise a nucleic acid encoding a variant of SEQ ID NO: 1 comprising a sequence at least about 70% or 75% or 80% or 85% or 90% or 95% or 97% or 98% or 99% identical to the sequence set forth in SEQ ID NO: 1 which retains the activity of osteoglycin, as described herein.

For example, the expression construct or vector may comprise a nucleic acid sequence which is at least 70% or 75% or 80% or 85% or 90% or 95% or 97% or 98% or 99% identical to the sequence set forth in SEQ ID NO: 2, provided that the nucleic acid encodes an osteoglycin protein having activity to enhance insulin action and/or increasing insulin secretion from pancreatic beta cells in response to an increase in glucose levels. In one example, the expression construct or vector comprises a nucleic acid sequence which is at least 70% identical to the sequence set forth in SEQ ID NO: 2. In one example, the expression construct or vector comprises a nucleic acid sequence which is at least 75% identical to the sequence set forth in SEQ ID NO: 2. In one example, the expression construct or vector comprises a nucleic acid sequence which is at least 80% identical to the sequence set forth in SEQ ID NO: 2. In one example, the expression construct or vector comprises a nucleic acid sequence which is at least 85% identical to the sequence set forth in SEQ ID NO: 2. In one example, the expression construct or vector comprises a nucleic acid sequence which is at least 90% identical to the sequence set forth in SEQ ID NO: 2. In one example, the expression construct or vector comprises a nucleic acid sequence which is at least 95% identical to the sequence set forth in SEQ ID NO: 2. In one example, the expression construct or vector comprises a nucleic acid sequence which is at least 97% identical to the sequence set forth in SEQ ID NO: 2. In one example, the expression construct or vector comprises a nucleic acid sequence which is at least 98% identical to the sequence set forth in SEQ ID NO: 2. In one example, the expression construct or vector comprises a nucleic acid sequence which is at least 99% identical to the sequence set forth in SEQ ID NO: 2. In one example, the expression construct or vector comprises a nucleic acid sequence set forth in SEQ ID NO: 2.

In another example, the expression construct or vector may comprise a nucleic acid encoding an osteoglycin protein from another mammalian species, such as a murine, a bovine, a canine, an equine, a feline, a porcine, a simian or other mammalian osteoglycin protein.

Methods for producing an expression constructs for expressing a protein of interest in a cell will be apparent to the skilled artisan. For example, the nucleic acid to be expressed in the cell i.e., a nucleic acid encoding osteoglycin protein, will be operably-linked to a promoter for inducing expression of the osteoglycin protein in the cell. For example, the nucleic acid will be linked to a promoter operable in a variety of cells of a subject, such as, for example, a viral promoter, e.g., a CMV promoter (e.g., a CMV-IE promoter) or a SV-40 promoter. Additional suitable promoters are known in the art and shall be taken to apply mutatis mutandis to the present example of the disclosure.

In one example, the nucleic acid will be prepared in the form of an expression construct. As used herein, the term “expression construct” refers to a nucleic acid that has the ability to confer expression on a nucleic acid to which it is operably connected, in a cell. Within the context of the present disclosure, it is to be understood that an expression construct may comprise or be a plasmid, bacteriophage, phagemid, cosmid, virus sub-genomic or genomic fragment, or other nucleic acid capable of maintaining and/or replicating heterologous DNA in an expressible format.

Methods for the construction of a suitable expression constructs of the disclosure will be apparent to the skilled artisan and are described, for example, in Ausubel et al (In: Current Protocols in Molecular Biology. Wiley Interscience, ISBN 047 150338, 1987) or Sambrook et al (In: Molecular Cloning: Molecular Cloning: A Laboratory Manual, Cold Spring Harbor Laboratories, New York, Third Edition 2001). For example, each of the components of the expression construct is amplified from a suitable template nucleic acid using, for example, PCR and subsequently cloned into a suitable expression construct, such as for example, a plasmid or a phagemid.

Vectors suitable for use with such an expression construct are known in the art and/or described herein. For example, an expression vector suitable for the method of the present disclosure in a mammalian cell is, for example, a vector of the pcDNA vector suite supplied by Invitrogen, a vector of the pCI vector suite (Promega), a vector of the pCMV vector suite (Clontech), a pM vector (Clontech), a pSI vector (Promega), a VP 16 vector (Clontech) or a vector of the pcDNA vector suite (Invitrogen).

The skilled artisan will be aware of additional vectors and sources of such vectors, such as, for example, Life Technologies Corporation, Clontech or Promega.

Means for introducing expression constructs and vectors into a cell for expression of a desired protein are also known to those skilled in the art. The technique used for a given organism depends on the known successful techniques. Means for introducing recombinant DNA into cells include microinjection, transfection mediated by DEAE-dextran, transfection mediated by liposomes such as by using lipofectamine (Gibco, Md., USA) and/or cellfectin (Gibco, Md., USA), PEG-mediated DNA uptake, electroporation and microparticle bombardment such as by using DNA—coated tungsten or gold particles (Agracetus Inc., Wis., USA) amongst others.

Alternatively, an expression construct of the disclosure is a viral vector. Suitable viral vectors are known in the art and commercially available. Conventional viral-based systems for the delivery of a nucleic acid and integration of that nucleic acid into a host cell genome include, for example, a retroviral vector, a lentiviral vector, or an adeno-associated viral vector. Alternatively, an adenoviral vector is useful for introducing a nucleic acid that remains episomal into a host cell. Viral vectors are an efficient and versatile method of gene transfer in target cells and tissues. Additionally, high transduction efficiencies have been observed in many different cell types and target tissues.

For example, a retroviral vector generally comprises cis-acting long terminal repeats (LTRs) with packaging capacity for up to 6-10 kb of foreign sequence. The minimum cis-acting LTRs are sufficient for replication and packaging of a vector, which is then used to integrate the expression construct into the target cell to provide long term expression. Widely used retroviral vectors include those based upon murine leukemia virus (MuLV), gibbon ape leukemia virus (GaLV), simian immunodeficiency virus (SrV), human immunodeficiency virus (HIV), and combinations thereof (see, e.g., Buchscher et al., J Virol. 56:2731-2739 (1992); Johann et al, J. Virol. 65:1635-1640 (1992); Sommerfelt et al, Virol. 76:58-59 (1990); Wilson et al, J. Virol. 63:274-2318 (1989); Miller et al., J. Virol. 65:2220-2224 (1991); PCT/US94/05700; Miller and Rosman BioTechniques 7:980-990, 1989; Miller, A. D. Human Gene Therapy 7:5-14, 1990; Scarpa et al Virology 75:849-852, 1991; Burns et al. Proc. Natl. Acad. Sci USA 90:8033-8037, 1993).

Various adeno-associated virus (AAV) vector systems have also been developed for nucleic acid delivery. AAV vectors can be readily constructed using techniques known in the art. See, e.g., U.S. Pat. Nos. 5,173,414 and 5,139,941; International Publication Nos. WO 92/01070 and WO 93/03769; Lebkowski et al. Molec. Cell. Biol. 5:3988-3996, 1988; Vincent et al. (1990) Vaccines 90 (Cold Spring Harbor Laboratory Press);Carter Current Opinion in Biotechnology 5:533-539, 1992; Muzyczka. Current Topics in Microbiol, and Immunol. 158:97-129, 1992; Kotin, Human Gene Therapy 5:793-801, 1994; Shelling and Smith Gene Therapy 7:165-169, 1994; and Zhou et al. J Exp. Med. 179:1867-1875, 1994.

Additional viral vectors useful for delivering an expression construct of the disclosure include, for example, those derived from the pox family of viruses, such as vaccinia virus and avian poxvirus or an alphavirus or a conjugate virus vector (e.g. that described in Fisher-Hoch et al., Proc. Natl Acad. Sci. USA 56:317-321, 1989).

### Functional Assays

Methods for determining the ability of an agent to increase the activity of osteoglycin in a subject, and thereby enhance insulin action and/or increase insulin sensitivity and/or increasing insulin secretion from pancreatic beta cells in response to an increase in glucose levels, will be apparent to the skilled person.

For example, the effect of an agent of the disclosure on insulin release and/or sensitivity can be determined by performing a glucose tolerance test on an animal and/or cell using methodologies known in the art e.g., as described in Stumvoll et al., (2000) Diabetes Care, 23(3):295-301.

An exemplary in vitro method for determining the effect of an agent of the disclosure is to contact β-cells (e.g., a β-cell line such as MIN6 or HC-9) with the agent and assess its effect, e.g., on insulin secretion, action and/or activity, such as in response to glucose stimulation. Exemplary assays for measuring insulin secretion are known in the art and include, for example commercially available enzyme-linked immunosorbent assays (ELISAs) as exemplified herein. Alternatively, or in addition, standard molecular assays configured to detect changes in expression levels of insulin genes, Ins1 and Ins2, may be used to measure insulin secretion. An agent that increases insulin secretion in response to glucose by increasing osteoglycin activity is considered to be suitable.

### Pharmaceutical Compositions

The agent for increasing ostoeglycin activity as disclosed herein (syn. active ingredient) is useful for parenteral, topical, oral, or local administration, aerosol administration, or transdermal administration, for prophylactic or for therapeutic treatment.

Formulation of the active ingredient to be administered will vary according to the agent, route of administration and formulation (e.g., solution, emulsion, capsule) selected. An appropriate pharmaceutical composition comprising the active ingredient to be administered can be prepared in a physiologically acceptable carrier. A mixture of active ingredients which increase ostoeglycin activity can also be used. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The active ingredient(s) of the disclosure can be lyophilized for storage and reconstituted in a suitable carrier prior to use according to art-known lyophilization and reconstitution techniques.

The optimum concentration of the active ingredient(s) in the chosen medium can be determined empirically, according to procedures known to the skilled artisan, and will depend on the ultimate pharmaceutical formulation desired.

The dosage ranges for the administration of the active ingredient are those large enough to produce the desired effect i.e., increase the activity of osteoglycin and thereby enhance insulin action and/or increase insulin secretion from pancreatic beta cells in response to an increase in glucose levels in a subject. For example, the composition comprises a therapeutically or prophylactically effective amount of the active ingredient(s).

As used herein, the term “effective amount” shall be taken to mean a sufficient quantity of the active ingredient to increase the activity of osteoglycin in a subject. The skilled artisan will be aware that such an amount will vary depending on, for example, the specific active ingredient used to achieve this effect and/or the particular subject and/or the type or severity of a condition being treated. Accordingly, this term is not to be construed to limit the disclosure to a specific quantity, e.g., weight or number of active ingredients, rather the present disclosure encompasses any amount of the the active ingredient that is sufficient to achieve the stated purpose. In one example, the “effective amount” is sufficient to increase the activity of osteoglycin in the subject and thereby enhance insulin action and/or increase insulin secretion from pancreatic beta cells in response to an increase in glucose levels in the subject.

As used herein, the term “therapeutically effective amount” shall be taken to mean a sufficient quantity of the antagonist to reduce or inhibit one or more symptoms of an abnormality of glucose metabolism and/or to increase GSIS in a subject.

As used herein, the term “prophylactically effective amount” shall be taken to mean a sufficient quantity of the antagonist to prevent or inhibit or delay the onset of one or more detectable symptoms of an abnormality of glucose metabolism.

The dosage should not be so large as to cause adverse side effects, such as hyper viscosity syndromes, pulmonary edema, congestive heart failure, and the like. Generally, the dosage will vary with the age, condition, sex and extent of the disease in the patient and can be determined by one of skill in the art. The dosage can be adjusted by the individual physician in the event of any complication. Dosage can vary from about 0.1 mg/kg to about 300 mg/kg, such as from about 0.2 mg/kg to about 200 mg/kg, for example from about 0.5 mg/kg to about 20 mg/kg, in one or more dose administrations daily, for one or several days.

One or more active ingrediatents of the present disclosure can be administered to an individual by an appropriate route, either alone or in combination with (before, simultaneous with, or after) another drug or agent.

It will be appreciated by those skilled in the art that some active ingredients (e.g., recombinant osteoglycin) of the present disclosure may be introduced into a subject by administering an expression construct of the disclosure or a cell expressing the active ingredient. A variety of methods can be used for introducing a nucleic acid encoding the active ingredient into a target cell in vivo. For example, the naked nucleic acid may be injected at the target site, may be encapsulated into liposomes, or may be introduced by way of a viral vector.

### Subjects to be Treated

In one example, a subject to be treated by the method of the present disclosure has impaired or inhibited GSIS. Methods for determining GSIS are known in the art and described, for example, in Fehse et al., J. Clin. Endocrinol and Metab., 90: 5991-5997, 2005. For example, subjects are fasted for about 8 hours and then (if required) insulin is infused to achieve a “normal” plasma glucose level (e.g., about 79-101 mg/dL). An intravenous glucose bolus is then administered and blood taken regularly to measure insulin levels. A subject that secretes less insulin in response to glucose compared to the mean level of secretion in a population of subjects known not to suffer from a glucose metabolism disorder is considered to suffer from GSIS.

In one example, the subject suffers from diabetes e.g., such as type 2 diabetes. For example, the subject has been diagnosed as having:
Fasting plasma glucose level≥7.0 mmol/l (126 mg/dl);
Plasma glucose ≥11.1 mmol/l (200 mg/dL) two hours after a 75 g oral glucose load as in a glucose tolerance test;
Symptoms of hyperglycemia and casual plasma glucose ≥11.1 mmol/l (200 mg/dl); and/or
Glycated hemoglobin (Hb A1C)≥6.5%.

In another example, the subject suffers from pre-diabetes. For example, the subject has been diagnosed as having:
Fasting blood sugar (glucose) level of:
   110 to 125 mg/dL (6.1 mM to 6.9 mM)—WHO criteria; or
   100 to 125 mg/dL (5.6 mM to 6.9 mM)—ADA criteria;
Two hour glucose tolerance test after ingesting the standardized 75 Gm glucose solution the blood sugar level of 140 to 199 mg/dL (7.8 to 11.0 mM); and/or
Glycated hemoglobin between 5.7 and 6.4 percent.

Combinations

An example of a compound that can be administered in a method of the disclosure or formulated with an agent of the disclosure is a GLP-1 analog or a GLP-1 receptor agonist.

In one example, the compound is GLP-1 or a GLP-1 receptor agonist. For example, GLP-1 comprises a sequence set forth in SEQ ID NO: 3. In one example, the GLP-1 is conjugated to or fused with albumin.

In one example, the compound is exenatide (comprising a sequence set forth in SEQ ID NO: 4). Exenatide is a 39-amino-acid peptide, an insulin secretagogue, with glucoregulatory effects.

In one example, the compound is exenatide LAR. Exenatide LAR comprises exenatide encapsulated in microspheres made of poly (D,L) lactic-co-glycolic acid.

In one example, the compound is liraglutide comprising a sequence set forth in

SEQ ID NO: 5 and having a C-16 fatty acid (palmitic acid) with a glutamic acid spacer on the lysine residue at position 26.

In one example, the compound is taspoglutide comprising a sequence set forth in SEQ ID NO: 6. Taspoglutide is a 8-(2-methylalanine)-35-(2-methylalanine)-36-L-argininamide derivative of the amino acid sequence 7-36 of human glucagon-like peptide 1.

In one example, the compound is albiglutide, which comprises two copies of a 30 amino acid sequence of human glucagon-like peptide 1 (GLP-1, fragment 7-36) that is DPP-IV resistant (by virtue of an alanine to glycine conversion at amino acid 8) fused with human albumin.

In one example, the compound is dulaglutide.

In one example, the compound is a DPPIV inhibitor.

In one example, the compound is sitagliptin as set out in Formula 13:

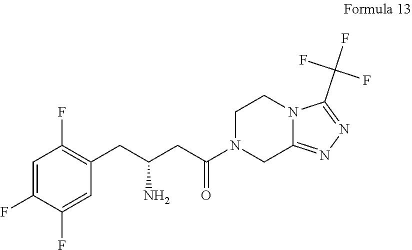 [SMILES: N[C@@H](CC(=O)N1CCn2c(nnc2C(F)(F)F)C1)Cc1cc(F)c(F)cc1F]

In one example, the compound is vildagliptin as set out in Formula 14:

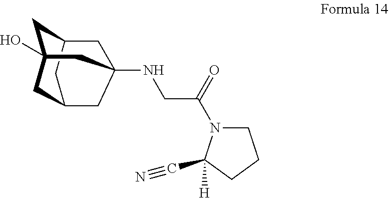

In one example, the compound is a sulphonylurea.

For example, the compound is glibenclamide as set out in Formula 15:

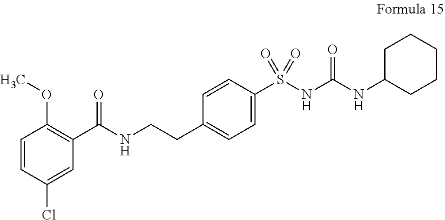 [SMILES: COc1ccc(Cl)cc1C(=O)NCCc1ccc(S(=O)(=O)NC(=O)NC2CCCCC2)cc1]

For example, the compound is glyburide as set out in Formula 16:

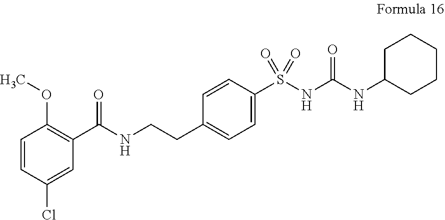 [SMILES: COc1ccc(Cl)cc1C(=O)NCCc1ccc(S(=O)(=O)NC(=O)NC2CCCCC2)cc1]

For example, the compound is gliclazide as set out in Formula 17:

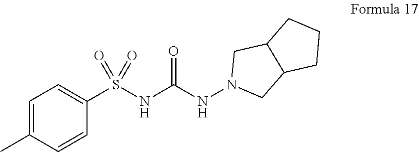 [SMILES: Cc1ccc(S(=O)(=O)NC(=O)NN2CC3CCCC3C2)cc1]

In one example, the compound is a meglitinide.

For example, the compound is repaglinide as set out in Formula 18:

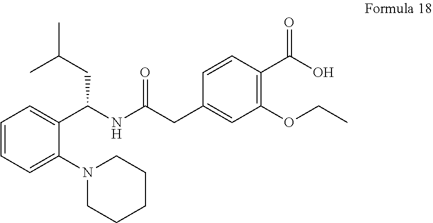 [SMILES: CCOc1cc(CC(=O)N[C@@H](CC(C)C)c2ccccc2N2CCCCC2)ccc1C(=O)O]

For example, the compound is nateglinide as set out in Formula 19:

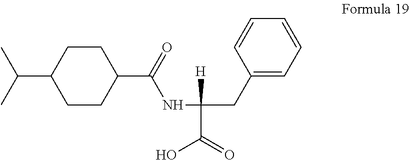 [SMILES: CC(C)C1CCC(C(=O)N[C@H](Cc2ccccc2)C(=O)O)CC1]

In one example, the compound is a GPR40 agonist.

For example, the compound is TAK-875 as set out in Formula 20:

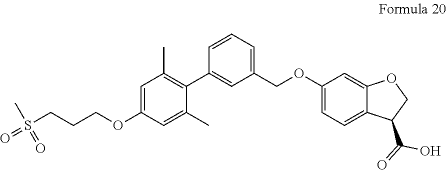 [SMILES: Cc1cc(OCCCS(C)(=O)=O)cc(C)c1-c1cccc(COc2ccc3c(c2)OC[C@H]3C(=O)O)c1]

For example, the compound is AMG-837 ((S)-3-(4-((4′-(trifluoromethyl)biphenyl-3-yl)methoxy)phenyl)hex-4-ynoic acid) as set out in Formula 21:

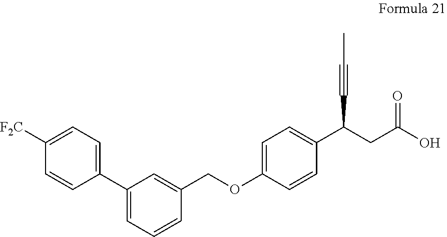

In one example, the compound is a GPR119 agonist.

For example, the compound is PSN632408 as set out in Formula 22:

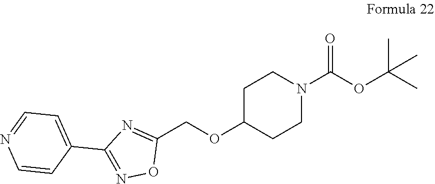 [SMILES: CC(C)(C)OC(=O)N1CCC(OCc2nc(-c3ccncc3)no2)CC1]

In one example, the compound is JNJ-38431055 as set forth in Formula 23

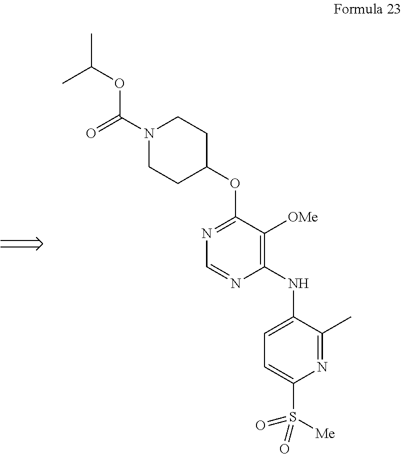 [SMILES: COc1c(Nc2ccc(S(C)(=O)=O)nc2C)ncnc1OC1CCN(C(=O)OC(C)C)CC1]

In one example, the compound is a glucokinase activator.

In one example, the compound is GKA50 as set forth in Formula 24

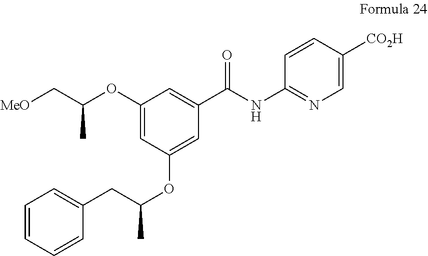 [SMILES: COC[C@H](C)Oc1cc(O[C@@H](C)Cc2ccccc2)cc(C(=O)Nc2ccc(C(=O)O)cn2)c1]

In one example, the compound is piragliatin as set forth in Formula 25:

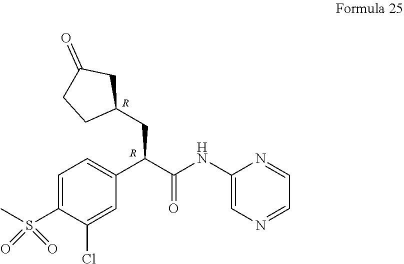

In one example, the compound is an inhibitor of the sodium glucose co-transporter-2 (SGLT-2). For example, the compound is empagliflozin, e.g., comprising a structure as set forth in Formula 26:

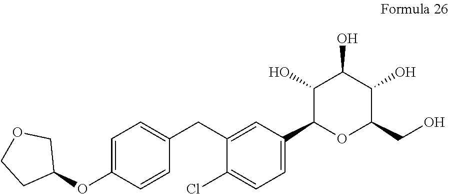 [SMILES: OC[C@H]1O[C@@H](c2ccc(Cl)c(Cc3ccc(O[C@H]4CCOC4)cc3)c2)[C@H](O)[C@@H](O)[C@@H]1O]

In another example, the compound is a thiazolidinone, such as, rosiglitazone, pioglitazone or troglitazone.

In another example, the compound is metformin.

In another example, the compound is an insulin analog e.g., insulin lispro, insulin aspart, insulin glulisine, insulin detemir, insulin degludec, insulin glargine or NPH insulin.

### Kits

The present disclosure additionally comprises a kit comprising one or more of the following:
(i) an agent which increases the activity of osteoglycin as disclosed herein; or
(ii) an agent which increases the activity of osteoglycin as disclosed herein and another compound, e.g., as described herein; and/or
(iii) a pharmaceutical composition of the disclosure.

In the case of a kit for therapeutic/prophylactic use, the kit can additionally comprise a pharmaceutically acceptable carrier or diluent.

Optionally a kit of the disclosure is packaged with instructions for use in a method described herein according to any example.

The present disclosure includes the following non-limiting Examples.

### EXAMPLES

In the work described in the following examples, the inventors identified a novel factor, osteoglycin, which acts as a mediator in a pathway originating in bone tissue that acts to alter whole body glucose homeostasis by affecting insulin secretion and insulin action. Based on this finding, the inventors sought to determine whether altering the activity of osteoglycin can improve glucose homeostasis and, in doing so, evaluate osteoglycin's potential as a treatment option for correcting glucose metabolism and/or homeostasis.

### Example 1

### Identifying Factors Participating in the Crosstalk Between Bone and Pancreas

In this example, the inventors set out to identify potential factors that were altered in their expression or released and participating in the crosstalk between bone and pancreas. In doing so, the inventors utilised a combination of microarray analysis as well as mass spectrometric analysis.

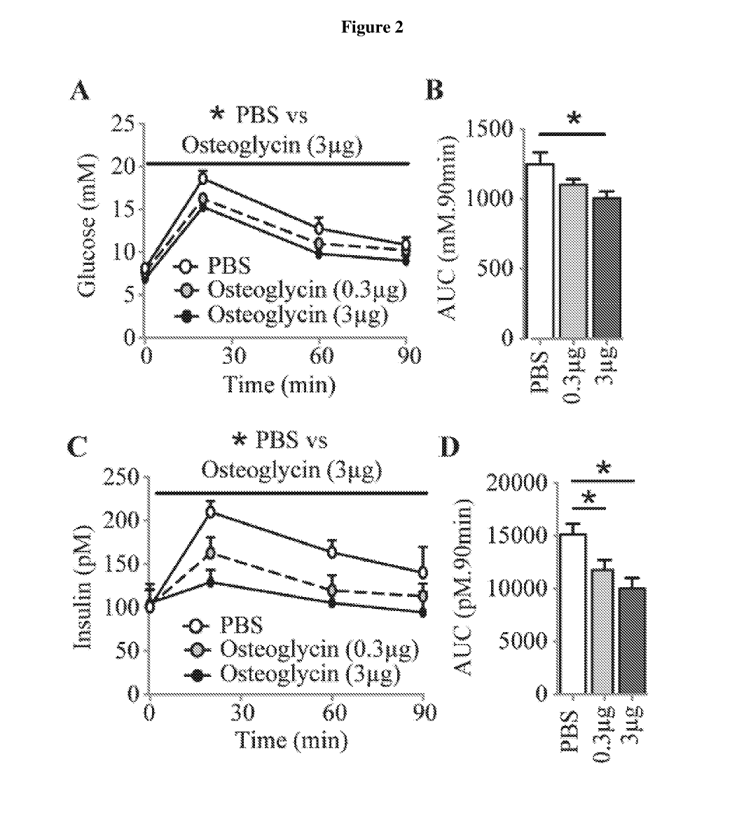

Briefly, microarray analysis was carried out on RNA from osteoblasts isolated by fluorescent activated cell sorting (FACS) utilising GFP reporter mice in which osteoblasts were identified by GFP expression driven by a 3.6 kb fragment of the α1(I)-collagen promotor (Kalajzic et al., (2002) J. Bone Miner. Res., 17:15-25). The GFP mice were bred to previously described germline Y1 receptor knockout mice (Y1^{-/-}) described in Howell et al., (2003) J. Neurochem., 86:646-659. In this regard, the inventors have previously shown that signalling via Y1 receptors in beta cells and osteoblasts is important to the control of bone mass and glucose homeostasis (Lee et al., (2015) Mol. Metab., 4: 164-174). FACS was then used to isolate GFP expressing osteoblasts from wild type (WT) and Y1-deficient mice (FIG. 2). The microarray data was analysed for changes in expression of proteins belonging to secretory pathways. Interestingly, one factor, osteoglycin, stood out with a significant 3.1-fold down regulation in expression in the Y1 deficient osteoblastic cells. This down-regulation was confirmed by qPCR (Fold change WT: 1.0±0.13; Y1^{-/-}: 0.4±0.03; p=0.04).

In order to confirm that this down regulation of expression in mRNA levels is also translated into actual protein changes, the inventors then performed mass spectrometry on osteoblastic cells (mineralising bone marrow stromal cells after 2 weeks in osteogenic media) isolated from mice lacking osteoblastic Y1 receptors and their wildtype littermates, and analysed the data for changes in secretory proteins. Importantly, when comparing this mass spectrometry data with the microarray analysis of Y1-deficient osteoblasts, osteoglycin was found to follow the same pattern in both analyses, with a 3.6-fold down-regulation in protein levels, as well as 3.1-fold down-regulated mRNA expression in Y1-deficient osteoblasts.

Based on these findings, the inventors hypothesised that osteoglycin may act as a humoral factor capable of regulating insulin and glucose homeostasis.

### Example 2

### Determining Effect of Osteoglycin on Whole Body Glucose Metabolism

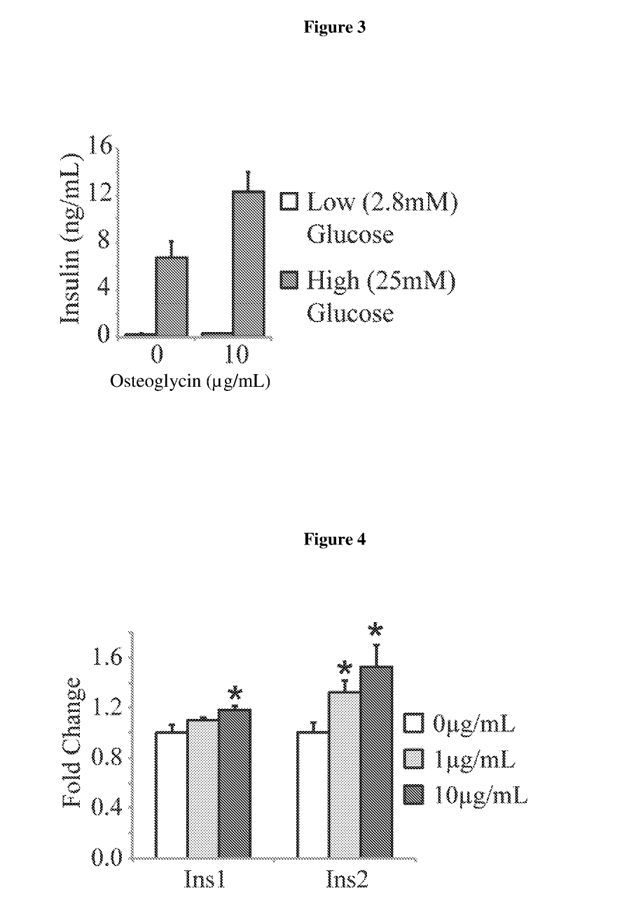

To investigate whether osteoglycin affects whole body glucose metabolism in vivo, intraperitoneal glucose tolerance tests were performed in 14 week old C57BL6 male mice treated with (i) 0.3 μg of osteoglycin in PBS (n=6), (ii) 3 μg of osteoglycin in PBS (n=6), or (iii) PBS only (n=6), four hours prior to performance of the glucose tolerance test. All treatment groups exhibited similar basal glucose levels before the glucose injection (FIG. 3A).

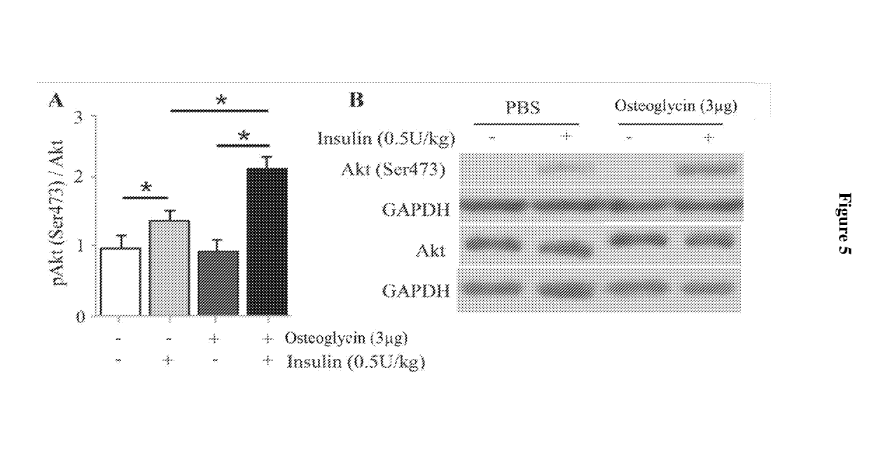

In response to administration of a glucose bolus (1 g/kg BW), mice treated with osteoglycin showed lower blood glucose levels over time and reduced area under the glucose curve compared to those of the vehicle group, significantly so for mice in the treatment group which received 3 μg of osteoglycin (FIG. 3A,B). Importantly, the improved glucose tolerance observed in mice treated with osteoglycin was associated with dose-dependent reductions in insulin levels and areas under the insulin curves versus those of vehicle group (FIG. 3C,D). These reductions in insulin were not due to a detrimental effect of osteoglycin on beta cell insulin secretion as evidenced by preliminary experiments showing a trend for osteoglycin treatment in vitro to improve insulin secretion from primary islets in response to high glucose levels (FIG. 4). Taken together, these data suggest that, under an acute setting, osteoglycin improves glucose tolerance due to enhanced insulin action and illustrates a mechanism by which NPY signalling in bone is regulating glucose homeostasis. These data suggest that the action of osteoglycin is at least 2-fold: firstly to directly affect pancreatic insulin secretion and secondly to enhance insulin action.

### Example 3

### Determining the Effect of Osteoglycin on Regulation of Pancreatic Insulin Secretion

In order to further confirm the ability of osteoglycin to directly regulate pancreatic insulin secretion, in vitro analyses were performed with pancreatic MIN6 cells.

Briefly, MIN6 cells were maintained in DMEM media containing 25 mM glucose, 10% FBS, 10 mM HEPES, 100 U/mL penicillin and 100 μg/mL streptomycin. For treatment experiments, cells were seeded at 4×10⁵ cells/mL, left to recover for 24 hours and then media was changed to DMEM low glucose media (5.5 mM) and 1% FBS for 24 hours prior to treatment. MIN6 cells were treated for 4 hours with recombinant mouse osteoglycin (R&D Systems) after which RNA was extracted from the cells followed by cDNA synthesis and qPCR.

Treatment of MIN6 cells with osteoglycin for 4 hours resulted in a significant, dose-dependent increase in the mRNA expression of both insulin genes, Ins1 and Ins2 (FIG. 5).

### Example 4

### Determining Mechanism by which Osteoglycin Enhances Insulin Action

To elucidate the underlying mechanism behind the ability of osteoglycin to enhance insulin action, the inventors investigated the influence of osteoglycin on Akt phosphorylation. Akt is a key enzyme involved in the insulin signalling pathway with phosphorylation and subsequent activation of this enzyme upon insulin stimulation directly linked to the regulation of glucose transport.

Briefly, male C57BL6 mice treated with (i) 3 μg of osteoglycin in PBS (n=6), or (ii) PBS only (n=6) four hours previously were administered insulin (0.5 U/kg BW) or saline by intraperitoneal injection, then 10 minutes later tissue was excised from the quadriceps muscle and Akt phosphorylation in the tissue was determined by Western Blot.

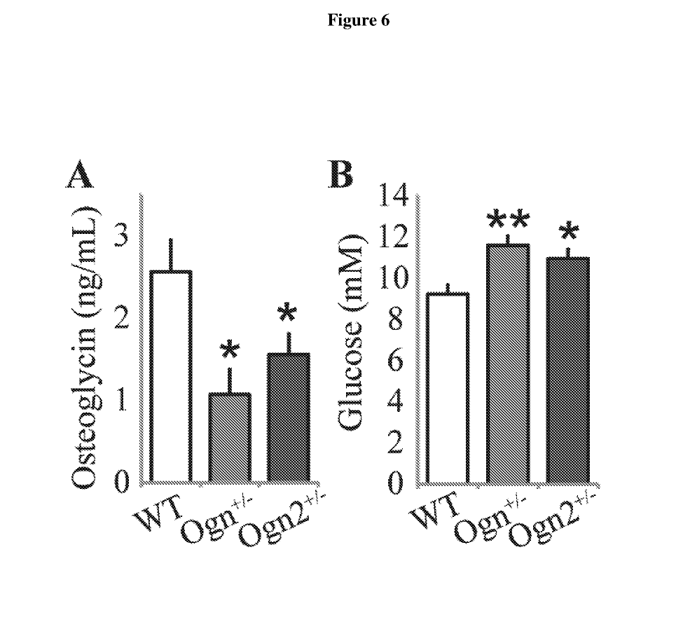

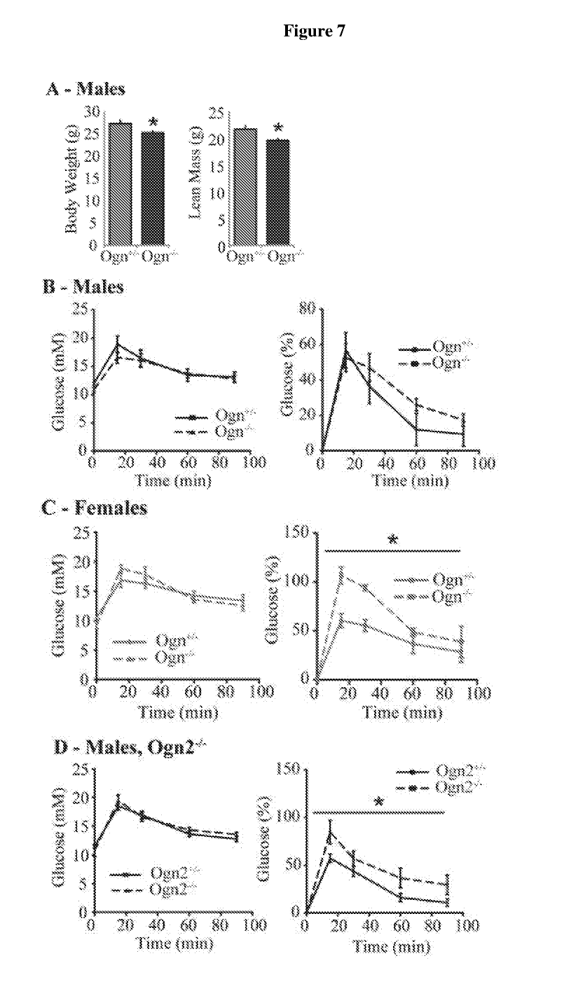

As expected, insulin stimulation significantly increased the Akt phosphorylation in PBS-treated mice (FIG. 6A,B). Notably, the insulin-induced Akt phosphorylation was significantly enhanced in mice pre-treated with osteoglycin (FIG. 7A,B), clearly demonstrating osteoglycin has a synergistic effect on insulin signalling in vivo, and through this mechanism can improve insulin action on target tissues such as muscle.

### Example 5

### Development and Testing of Osteoglycin Knockout Mice

Using CRISPR technology described in Ran et al., (2013) Cell, 154:1380-1389, the inventors targeted the first exon of osteoglycin and generated two separate knockout mouse lines (Ogn3^{-/-} and Ogn4^{-/-}). The Ogn3^{-/-} line has a 20 base pair deletion resulting in a premature stop codon, whereas the Ogn4^{-/-} line has a 21 base pair deletion which does not alter the subsequent reading frame.

To confirm the efficacy of the models, circulating levels of osteoglycin in the serum of 16 week old heterozygous male Ogn3^{+/-} and Ogn4^{+/-} mice were measured using a commercial ELISA kit (Wuhan USCN Business Co., Ltd.) according to the manufacturer's instructions.

Heterozygous mice from both lines displayed approximately a 50% reduction in serum levels of osteoglycin compared to wildtype controls (FIG. 7A). Interestingly, it was found that loss of only one allele of osteoglycin is sufficient to significantly raise circulating glucose levels in both lines (FIG. 7B).

Glucose tolerance tests were then performed for a cohort of heterozygous and homozygous mice from both lines (n=6 per group) using standard methodologies.

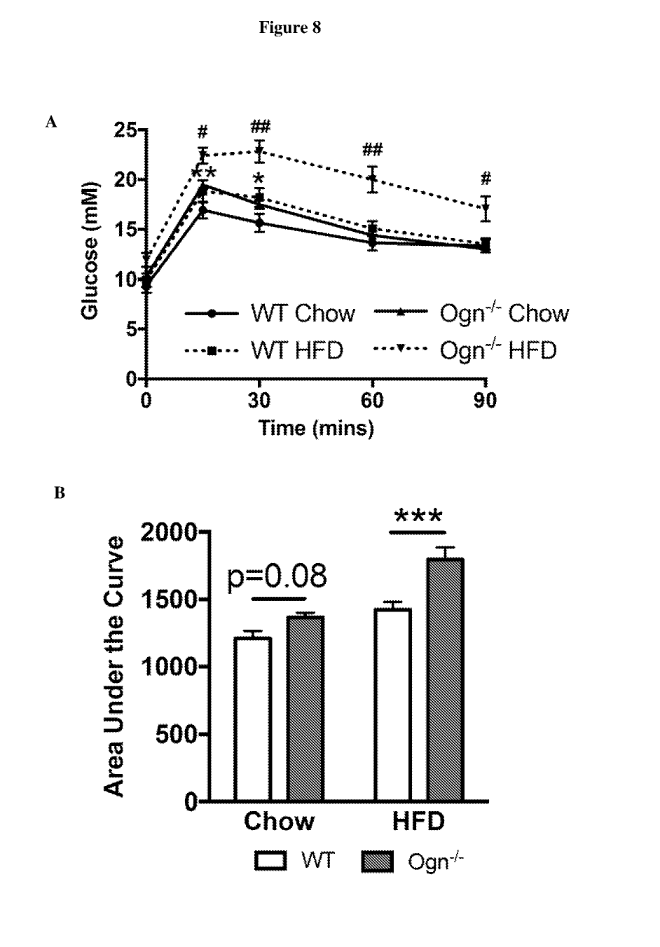

Data from the glucose tolerance tests indicated that male Ogn3^{-/-} mice have significantly lower body weight and lean mass than their heterozygous counterparts with a non-significant trend towards impaired clearance of glucose during glucose tolerance testing (FIG. 8A-B). However, the impairment in glucose clearance was more marked in female Ogn3^{-/-} mice and male Ogn4^{-/-} mice which both display significantly higher glucose levels than heterozygous mice throughout the glucose tolerance test.

### Example 6

### Testing of Osteoglycin Knockout Mice

Male Ogn−/− mice described in Example 5 and their wildtype littermates were fed either chow or high fat diet (HFD) (n=8-12 mice per group) and monitored for body weight from weaning onwards. At 11 weeks of age, the mice underwent a glucose tolerance test (GTT) following standard protocols using a dose of 1.0 g/kg. Sera collected during GTTs was assayed for glucose and insulin using a glucose oxidase assay kit (Trace Scientific) and an insulin radioimmunoassay (Merck Millipore), respectively.

Body composition of the Ogn−/− mice and their wildtype littermates was also analysed using a dedicated mouse dual energy X-ray absorptiometer (DXA) (Lunar Piximus II, GE Medical Systems, Madison Wis.) at 15 weeks, one week prior to cull. At 16 weeks, all animals were sacrificed, at which time the femora were dissected and micro-computed tomography (uCT) analysis was performed using a Skyscan 1174 scanner and analysis software (Skyscan, Aartselaar, Belgium) to examine 3-dimensional cortical bone structure and trabecular bone microstructure. Distal femora were scanned at 4.37 um pixel size. The image projections were reconstructed using NRecon software (Skyscan) and the reconstructed images were aligned using Dataviewer software (Skyscan). Trabecular bone mass was analysed across a 1 mm portion, located 0.5 mm from the growth plate of the femur whilst cortical bone mass was analysed across a 1 mm portion, located 2.5 mm from the growth plate of the femur. Regions of interest (ROIs) were drawn or automated to separate cortical and trabecular bone mass. The ROIs were then subsequently converted to binary images for volumetric and structural analyses using BatMan (Skyscan).

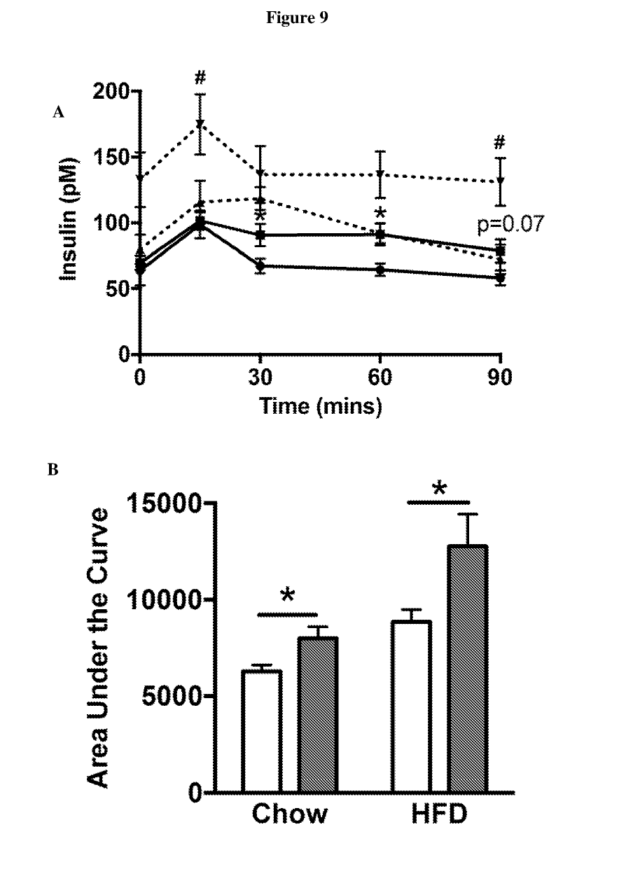

Ogn^{-/-} mice were found to have significantly impaired glucose clearance during a glucose tolerance test (GTT) under both chow and HFD fed conditions as shown by higher glucose levels (FIGS. 8A and 8B) and associated higher insulin levels (FIGS. 9A and 9B).

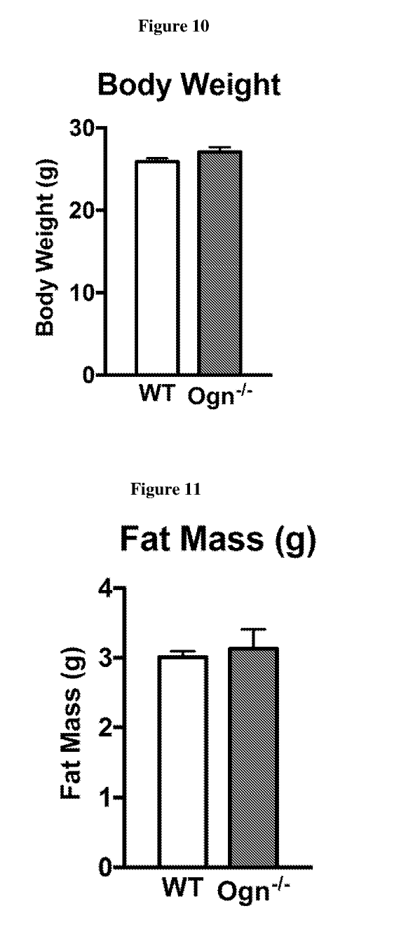

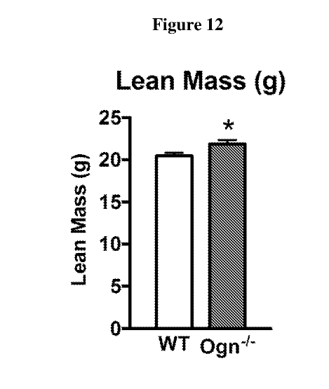

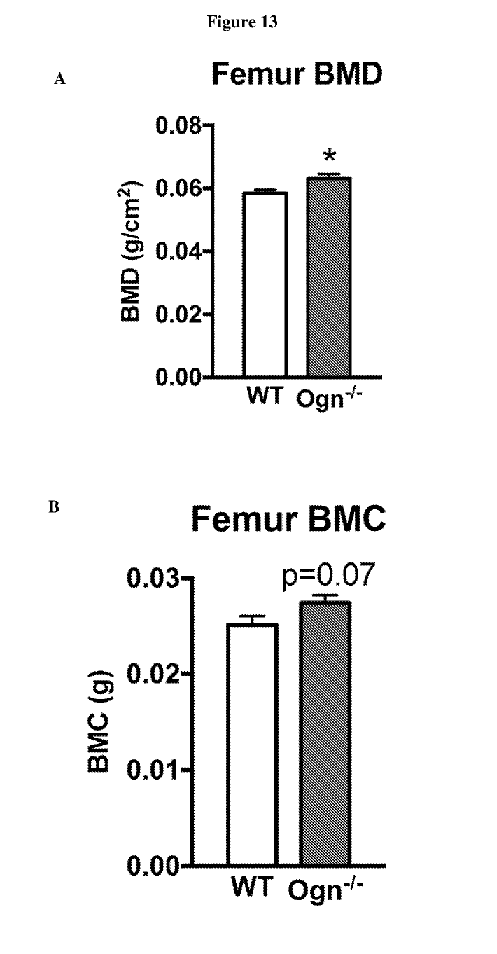

Despite showing no difference in body weight (FIG. 10) or fat mass (FIG. 11) relative to their wildtype littermates, Ogn^{-/-} mice showed significantly increased lean mass (FIG. 12) as well as femur bone mineral density (BMD) and bone mineral content (BMC) (FIGS. 13A and 13B, respectively) as measured by dual x-ray densitometry (DXA).

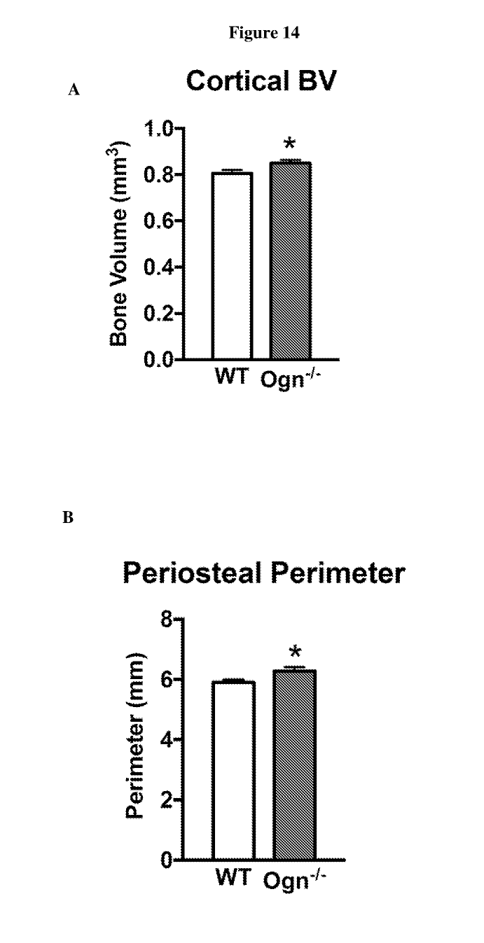

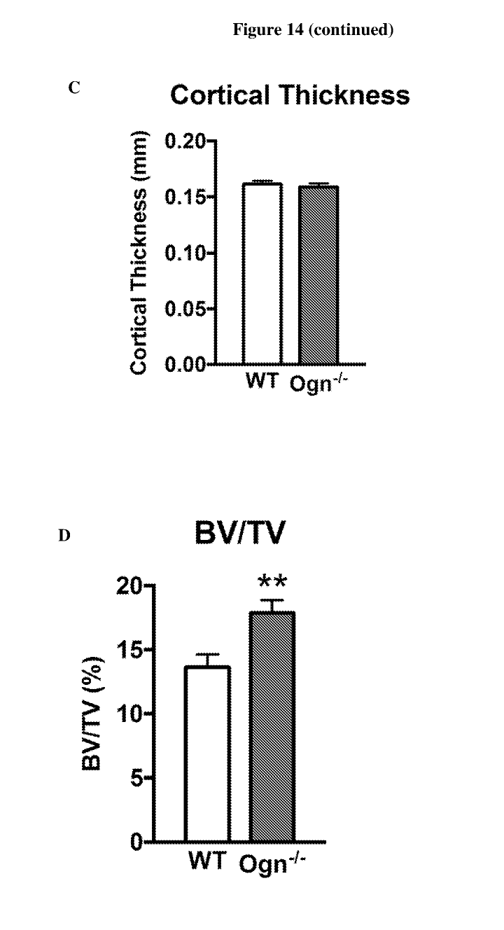

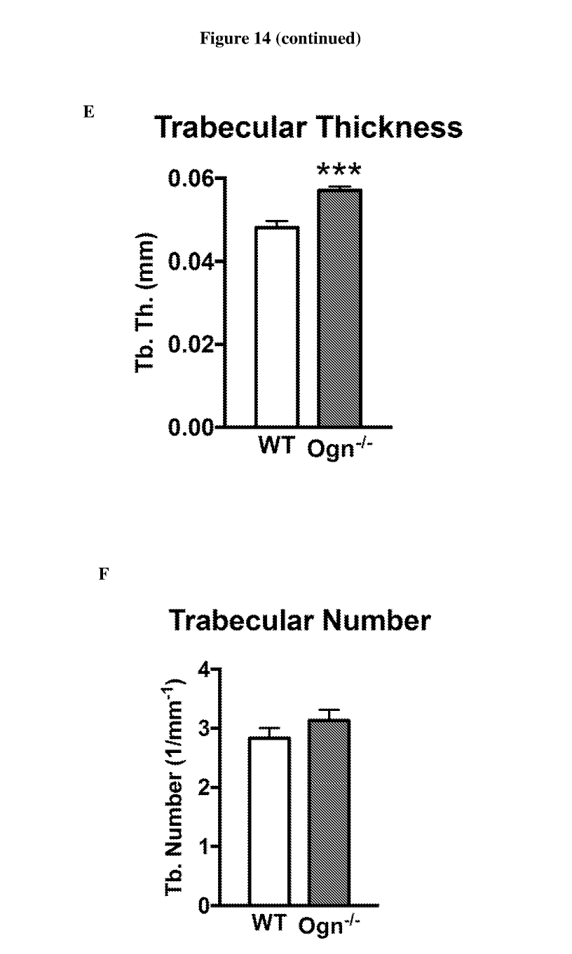

Micro cT analysis revealed that, relative to their wildtype littermates, male Ogn^{-/-} mice had significant increases in both cortical and trabecular bone in the femur as shown by significant increases in cortical bone volume (BV) (FIG. 14A) and periosteal perimeter (FIG. 14B) with no change in cortical thickness (FIG. 14C), and significant increases in trabecular bone volume (BV/TV) (FIG. 14D) and trabecular thickness (FIG. 14E) with no change in trabecular number (FIG. 14F).

### Example 7

### Measuring Circulating Levels of Osteoglycin

Serum osteoglycin levels were measured in a cohort of human patients including (i) individuals with type 2 diabetes, (ii) obese/overweight individuals and (iii) and lean individuals.

Serum osteoglycin levels were also measured in 16 week old wildtype mice fed a chow or high fat diet for 8 weeks.

Briefly, sera was collected and circulating osteoglycin levels measures using the method described in Chaudhuri et al., (2013) Diabetologia, 56(4):875-85 and an ELISA kit (USCN).

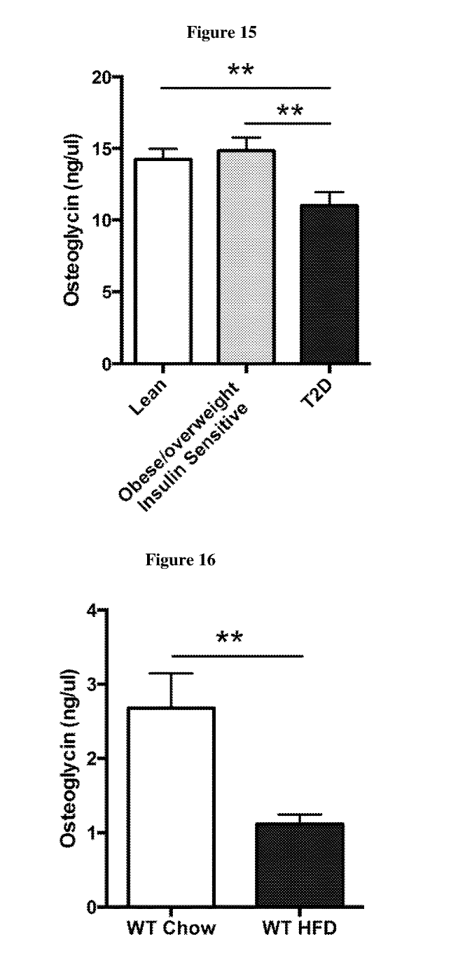

Circulating levels of osteoglycin were significantly lower in patients with type 2 diabetes (T2D) as compared to both lean and obese/overweight but insulin sensitive controls (FIG. 15).

A similar decrease in circulating levels of osteoglycin was seen in 16 week old male wildtype (WT) mice fed a HFD for 8 weeks compared to those fed chow (FIG. 16).

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method of treating or preventing an abnormality of glucose metabolism in a subject, said method comprising increasing the activity of osteoglycin in the subject.

2. A method for increasing glucose stimulated insulin secretion in a subject having reduced or impaired glucose-stimulated insulin secretion (GSIS), said method comprising increasing the activity of osteoglycin in the subject.

3. The method according to claim 1 or 2, wherein the increase in activity of osteoglycin is sufficient to enhance insulin action in the subject and thereby improve glucose tolerance of the subject.

4. The method according to any one of claims 1 to 3, wherein the increase in activity of osteoglycin is sufficient to increase insulin secretion from pancreatic beta cells in the subject in response to an increase in glucose levels in the subject.

5. The method according to any one of claims 1 to 4, wherein the subject suffers from an abnormality of glucose metabolism.

6. The method according to any one of claims 1 to 5, wherein the subject suffers from a condition selected from the group consisting of type 2 diabetes, obesity, hyperglycaemia and combinations thereof.

7. The method according to any one of claims 1 to 6, wherein the subject suffers from type 2 diabetes or is at risk of developing type 2 diabetes.

8. The method according to any one of claims 1 to 7, wherein the increase in osteoglycin activity is achieved by administering one or more agents select from:
(i) osteoglycin or a functional fragment, analog or derivative thereof to the subject; and/or
(ii) an expression vector comprising a nucleic acid encoding osteoglycin or a functional fragment thereof, wherein said expression vector is capable of expressing osteoglycin or a functional fragment thereof in the subject.

9. The method of claim 8, wherein the expression vector is a plasmid.

10. The method according to claim 8 or 9, wherein the agent(s) is/are administered in the form of a pharmaceutical composition.

11. The method of claim 10, wherein the pharmaceutical composition is administered a plurality of times to thereby maintain glucose homeostasis in the subject.

12. The method of claim 10 or 11, wherein the pharmaceutical composition comprises, or is administered concurrently with or concomitantly with, another therapeutic compound for treatment of a metabolic condition.

13. The method of claim 12, wherein the other therapeutic compound is glucagon like peptide 1 (GLP-1), a GLP-1 analog, a GLP-1 receptor agonist, a dipeptidyl peptidase 4 (DPPIV) inhibitor, a sulphonurea, a meglitinide, a GPR40 agonist, a GPR119 agonist, a sodium glucose co-transporter-2 inhibitor, a thiazolidinone, metformin, a glucokinase activator or an insulin analog.

14. The method of claim 13, wherein:
(i) the GLP-1 analog or GLP-1 receptor agonist is selected from the group consisting of exenatide, liraglutide, exenatide LAR, taspoglutide, albiglutide, dulaglutide and GLP1 conjugated to albumin; and/or
(ii) the DPPIV inhibitor is selected from the group consisting of sitagliptin (JANUVIA) and vidagliptin (GALVUS); and/or
(iii) the sulphonylurea is selected from the group consisting of glibenclamide, glyburide and gliclazide; and/or
(iv) the meglitinide is selected from the group consisting of repaglinide and nateglinide; and/or
(v) the GPR40 agonist is selected from the group consisting of TAK-875 and AMG-837; and/or
(vi) the GPR119 agonist is selected from the group consisting of PSN632408, JNJ-38431055; and/or
(vii) the glucokinase activator is selected from the group consisting of GKA50, piragliatin (RO4389620) and ZYGK1; and/or
(viii) the sodium glucose co-transporter-2 inhibitor is empagliflozin; and/or
(ix) the thiazolidinone is rosiglitazone, pioglitazone or troglitazone; and/or
(x) the insulin analog is insulin lispro, insulin aspart, insulin glulisine, insulin detemir, insulin degludec, insulin glargine or NPH insulin.

15. Use of an agent which increases activity of osteoglycin in a subject in the preparation of a medicament for treatment or prevention of abnormal glucose metabolism in a subject in need thereof.

16. Use of an agent which increases activity of osteoglycin in the preparation of a medicament for treatment or prevention of a condition associated with reduced or impaired glucose-stimulated insulin secretion (GSIS).

17. The use according to claim 15 or 16, wherein the agent is present in an amount effective to enhance insulin action in the subject and thereby improve glucose tolerance of the subject.

18. The use according to any one of claims 15 to 16, wherein the agent is present in an amount effective to increase insulin secretion from pancreatic beta cells in the subject in response to an increase in glucose levels.

19. The use according to any one of claims 15 to 18, wherein the medicament is for treatment or prevention of a condition selected from the group consisting of type 2 diabetes, obesity, hyperglycaemia and combinations thereof.

20. The use according to any one of claims 15 to 19, wherein the medicament is for treatment or prevention of type 2 diabetes.

21. The use according to any one of claims 15 to 20, wherein the agent which increases activity of osteoglycin is selected from:
(i) osteoglycin or a functional fragment, analog or derivative thereof; and/or
(ii) an expression vector comprising a nucleic acid encoding osteoglycin and which is capable of expressing osteoglycin or a functional fragment thereof in a cell.

22. The use of claim 21, wherein the expression vector is a plasmid.

23. The use according to any one of claims 15 to 22, wherein the medicament comprises another therapeutic compound for treatment of a metabolic condition.

24. The use of claim 23, wherein the other therapeutic compound is glucagon like peptide 1 (GLP-1), a GLP-1 analog, a GLP-1 receptor agonist, a dipeptidyl peptidase 4 (DPPIV) inhibitor, a sulphonurea, a meglitinide, a GPR40 agonist, a GPR119 agonist, a sodium glucose co-transporter-2 inhibitor, a thiazolidinone, metformin, a glucokinase activator or an insulin analog.

25. The use of claim 24, wherein:
(i) the GLP-1 analog or GLP-1 receptor agonist is selected from the group consisting of exenatide, liraglutide, exenatide LAR, taspoglutide, albiglutide, dulaglutide and GLP1 conjugated to albumin; and/or
(ii) the DPPIV inhibitor is selected from the group consisting of sitagliptin (JANUVIA) and vidagliptin (GALVUS); and/or
(iii) the sulphonylurea is selected from the group consisting of glibenclamide, glyburide and gliclazide; and/or
(iv) the meglitinide is selected from the group consisting of repaglinide and nateglinide; and/or
(v) the GPR40 agonist is selected from the group consisting of TAK-875 and AMG-837; and/or
(vi) the GPR119 agonist is selected from the group consisting of PSN632408, JNJ-38431055; and/or
(vii) the glucokinase activator is selected from the group consisting of GKA50, piragliatin (RO4389620) and ZYGK1; and/or
(viii) the sodium glucose co-transporter-2 inhibitor is empagliflozin; and/or
(ix) the thiazolidinone is rosiglitazone, pioglitazone or troglitazone; and/or
(x) the insulin analog is insulin lispro, insulin aspart, insulin glulisine, insulin detemir, insulin degludec, insulin glargine or NPH insulin.

26. A composition comprising an agent which increases activity of osteoglycin for treatment or prevention of abnormal glucose metabolism in a subject in need thereof.

27. A composition comprising an agent which increases activity of osteoglycin for treatment or prevention of a condition associated with reduced or impaired glucose-stimulated insulin secretion (GSIS).

28. The composition according to claim 26 or 27, wherein the agent is present in an amount effective to enhance insulin action in the subject and thereby improve glucose tolerance of the subject.

29. The composition according to any one of claims 26 to 28, wherein the agent is present in an amount effective to increase insulin secretion from pancreatic beta cells in the subject in response to an increase in glucose levels.

30. The composition according to any one of claims 26 to 29 for treatment or prevention of a condition selected from the group consisting of type 2 diabetes, obesity, hyperglycaemia and combinations thereof.

31. The composition according to any one of claims 26 to 30 for treatment or prevention of type 2 diabetes.

32. The composition according to any one of claims 26 to 31, wherein the agent which increases activity of osteoglycin is selected from:
(i) osteoglycin or a functional fragment, analog or derivative thereof; and/or
(ii) an expression vector comprising a nucleic acid encoding osteoglycin and which is capable of expressing osteoglycin or a functional fragment thereof in a cell.

33. The composition of claim 32, wherein the expression vector is a plasmid.

34. The composition according to any one of claims 26 to 33, wherein the composition comprises a pharmaceutically acceptable carrier, diluent or excipient.

35. The composition according to any one of claims 26 to 34, wherein the composition comprises another therapeutic compound for treatment of a metabolic condition.

36. The composition of claim 35, wherein the other therapeutic compound is glucagon like peptide 1 (GLP-1), a GLP-1 analog, a GLP-1 receptor agonist, a dipeptidyl peptidase 4 (DPPIV) inhibitor, a sulphonurea, a meglitinide, a GPR40 agonist, a GPR119 agonist, a sodium glucose co-transporter-2 inhibitor, a thiazolidinone, metformin, a glucokinase activator or an insulin analog.

37. The composition of claim 36, wherein:
(i) the GLP-1 analog or GLP-1 receptor agonist is selected from the group consisting of exenatide, liraglutide, exenatide LAR, taspoglutide, albiglutide, dulaglutide and GLP1 conjugated to albumin; and/or
(ii) the DPPIV inhibitor is selected from the group consisting of sitagliptin (JANUVIA) and vidagliptin (GALVUS); and/or
(iii) the sulphonylurea is selected from the group consisting of glibenclamide, glyburide and gliclazide; and/or
(iv) the meglitinide is selected from the group consisting of repaglinide and nateglinide; and/or
(v) the GPR40 agonist is selected from the group consisting of TAK-875 and AMG-837; and/or
(vi) the GPR119 agonist is selected from the group consisting of PSN632408, JNJ-38431055; and/or
(vii) the glucokinase activator is selected from the group consisting of GKA50, piragliatin (RO4389620) and ZYGK1; and/or
(viii) the sodium glucose co-transporter-2 inhibitor is empagliflozin; and/or
(ix) the thiazolidinone is rosiglitazone, pioglitazone or troglitazone; and/or
(x) the insulin analog is insulin lispro, insulin aspart, insulin glulisine, insulin detemir, insulin degludec, insulin glargine or NPH insulin.


SEQUENCE LISTING (6 sequences)

SEQ ID NO: 1 - protein, 298 aa, Homo sapiens
MKTLQSTLLLLLLVPLIKPAPPTQQDSRIIYDYGTDNFEESIFSQDYEDKYLDGKNIKEKETVIIPNEKSLQLQKDEAITPLPPKKENDEMPTCLLCVCLSGSVYCEEVDIDAVPPLPKESAYLYARFNKIKKLTAKDFADIPNLRRLDFTGNLIEDIEDGTFSKLSLLEELSLAENQLLKLPVLPPKLTLFNAKYNKIKSRGIKANAFKKLNNLTFLYLDHNALESVPLNLPESLRVIHLQFNNIASITDDTFCKANDTSYIRDRIEEIRLEGNPIVLGKHPNSFICLKRLPIGSYF

SEQ ID NO: 2 - DNA, 2786 bp, Homo sapiens
cacatggaaactgttcttaaagctgctgggccctgaaattttactcagcagtttgaaatcaagacatagcttttctcattcaccctcccacttggggctaatgcacagacatgaacatctattgaggaaaaccacaaaaaacttcaaaacagctacaacgggaaaaagagagttttgtcccacagtcagcaggccactagtttattaacttccagtcaccttgatttttgctaaaatgaagactctgcagtctacacttctcctgttactgcttgtgcctctgataaagccagcaccaccaacccagcaggactcacgcattatctatgattatggaacagataattttgaagaatccatatttagccaagattatgaggataaatacctggatggaaaaaatattaaggaaaaagaaactgtgataatacccaatgagaaaagtcttcaattacaaaaagatgaggcaataacaccattacctcccaagaaagaaaatgatgaaatgcccacgtgtctgctgtgtgtttgtttaagtggctctgtatactgtgaagaagttgacattgatgctgtaccacccttaccaaaggaatcagcctatctttacgcacgattcaacaaaattaaaaagctgactgccaaagattttgcagacatacctaacttaagaagactcgattttacaggaaatttgatagaagatatagaagatggtactttttcaaaactttctctgttagaagaactttcacttgctgaaaatcaactactaaaacttccagttcttcctcccaagctcactttatttaatgcaaaatacaacaaaatcaagagtaggggaatcaaagcaaatgcattcaaaaaactgaataacctcaccttcctctacttggaccataatgccctggaatccgtgcctcttaatttaccagaaagtctacgtgtaattcatcttcagttcaacaacatagcttcaattacagatgacacattctgcaaggctaatgacaccagttacatccgggaccgcattgaagagatacgcctggagggcaatccaatcgtcctgggaaagcatccaaacagttttatttgcttaaaaagattaccgatagggtcatacttttaacctctattggtacaacatataaatgaaagtacacctacactaatagtctgtctcaacaatgagtaaaggaacttaagtattggtttaatattaaccttgtatctcattttgaaggaatttaatattttaagcaaggatgttcaaaatcttacatataataagtaaaaagtaagactgaatgtctacgttcgaaacaaagtaatatgaaaatatttaaacagcattacaaaatcctagtttatactagactaccatttaaaaatcatgtttttatataaatgcccaaatttgagatgcattattcctattactaatgatgtaagtacgaggataaatccaagaaactttcaactctttgcctttcctggcctttactggatcccaaaagcatttaaggtacatgttccaaaaactttgaaaagctaaatgtttcccatgatcgctcattcttcttttatgattcatacgttattccttataaagtaagaactttgttttcctcctatcaaggcagctattttattaaatttttcacttagtctgagaaatagcagatagtctcatatttaggaaaactttccaaataaaataaatgttattctctgataaagagctaatacagaaatgttcaagttattttactttctggtaatgtcttcagtaaaatattttctttatctaaatattaacattctaagtctaccaaaaaaagttttaaactcaagcaggccaaaaccaatatgcttataagaaataatgaaaagttcatccatttctgataaagttctctatggcaaagtctttcaaatacgagataactgcaaaatattttccttttatactacagaaatgagaatctcatcaataaattagttcaagcataagatgaaaacagaatattctgtggtgccagtgcacactaccttcccacccatacacatccatgttcactgtaacaaactgaatattcacaataaagcttctgagtaacactttctgattactcatgataaactgacatggctaactgcaagaattaaatcttctatctgagagtaataatttatgatgactcagtggtgccagagtaaagtttctaaaataacattcctctcacttgtaccccactaaaagtattagactacacattacattgaagttaaacacaaaattatcagtgttttagaaacatgagtccggactgtgtaagtaaaagtacaaacattatttccaccataaagtatgtattgaaatcaagttgtctctgtgtacagaatacatacttattcccatttttaagcatttgcttctgttttccctacctagaatgtcagatgtttttcagttatctccccatttgtcaaagttgacctcaagataacatttttcattaaagcatctgagatctaagaacacaattattattctaacaatgattattagctcattcacttattttgataactaatgatcacagctattatactactttctcgttattttgtgtgcatgcctcatttccctgacttaaacctcactgagagcgcaaaatgcagctttatactttttactttcaattgcctagcacaatagtgagtacatttgaattgaatatataataaatattgcaaaataaaatccatctaaatagaaa

SEQ ID NO: 3 - protein, 31 aa, Artificial sequence
  Human GLP-1
HAEGTFTSDVSSYLEGQAAKEFIAWLVKGRG

SEQ ID NO: 4 - protein, 39 aa, Artificial sequence
  Exenatide
HGEGTFTSDLSKQMEEEAVRLFIEWLKNGGPSSGAPPPS

SEQ ID NO: 5 - protein, 31 aa, Artificial sequence; identical to SEQ ID NO: 3
  Liraglutide
  MISC_FEATURE (26)..(26): Conjugated C-16 fatty acid (palmitic acid) with a glutamic acid spacer

SEQ ID NO: 6 - protein, 30 aa, Artificial sequence
  Taspoglutide
  MISC_FEATURE (2)..(2): Ala at position 2 is 2-methyl-Ala
  MISC_FEATURE (29)..(29): Ala at position 2 is 2-methyl-Ala
HAEGTFTSDVSSYLEGQAAKEFIAWLVKAR